(19) United States
(12) Patent Application Publication
(10) Pub. No.: US 2021/0077312 A1
(43) Pub. Date: Mar. 18, 2021

(54) DIAPER FOR SENSING URINE AND FECES, URINE- AND FECES-SENSING SHEET FOR DIAPER, AND URINE- AND FECES-SENSING DEVICE

(71) Applicant: KOREA UNIVERSITY RESEARCH AND BUSINESS FOUNDATION, Seoul (KR)

(72) Inventors: Kwonhee KIM, Seoul (KR); Soowon CHAE, Seoul (KR)

(21) Appl. No.: 17/050,254

(22) PCT Filed: Apr. 2, 2019
(86) PCT No.: PCT/KR2019/003874

§ 371 Date: Oct. 23, 2020

(30) Foreign Application Priority Data
May 24, 2018 (KR) 10-2018-0059316
Aug. 7, 2018 (KR) 10-2018-0091986
Aug. 17, 2018 (KR) 10-2018-0096363
Aug. 17, 2018 (KR) 10-2018-0096364
Aug. 17, 2018 (KR) 10-2018-0096365
Aug. 17, 2018 (KR) 10-2018-0096366
Aug. 17, 2018 (KR) 20-2018-0003857
Dec. 31, 2018 (KR) 10-2018-0173949
Dec. 31, 2018 (KR) PCT/KR2018/016985
Apr. 1, 2019 (KR) 10-2019-0038022

(51) Int. Cl. A61F 13/42; A61F 13/49; A61F 13/15; A61F 13/44

(52) U.S. Cl. CPC A61F 13/42; A61F 13/44; A61F 13/15585; A61F 13/49

(57) ABSTRACT

Disclosed is a diaper for sensing urine and feces including a waterproof cover, an absorbing layer located on the inner side of the waterproof cover, and a lining layer located on the inner side of the absorbing layer, wherein the lining layer includes multiple conductive sensing lines positioned on the inner surface of the lining layer that makes contact with the human body, wherein the conductive sensing lines are arranged spaced apart from each other, wherein at least one of the electrical resistance, capacitance, and inductance between the conductive sensing lines changes upon contact between the conductive sensing lines and urine or feces.

### TECHNICAL FIELD

Embodiments of the present disclosure relate to a diaper, a urine- and feces-sensing sheet for a diaper, and a urine- and feces-sensing device. More specifically, embodiments of the present disclosure relate to a diaper for sensing urine or feces to sense change in the diaper caused by the urine or feces, a urine- and feces-sensing sheet for a diaper that is attached to a conventional diaper and implements a urine- and feces-sensing function, and a urine- and feces-sensing device usable in connection with the diaper and the sheet.

### BACKGROUND ART

In general, diapers can be classified into a band type diaper, a panties type diaper, a pad type diaper, etc., depending on an application purpose, or a diaper for an infant and a diaper for an adult depending on a type of a wearer. The diaper can be composed of three layers: a lining layer, an absorbing layer and a waterproof cover.

### DETAILED DESCRIPTION OF THE INVENTION

### Technical Problem

A purpose of the present disclosure is to provide a diaper for an infant or an adult which is capable of sensing absence or presence of urine or feces of a diaper wearer by a conductive sensing line including a material with electrical conductivity through electrical change sensed upon contact with the urine or feces, a urine- and feces-sensing sheet which attaches to a conventional diaper without a urine- and feces-sensing function to implement the urine- and feces-sensing function, and a urine- and feces-sensing device usable in connection with the diaper and the sheet.

The technical tasks to be achieved by the present disclosure are not limited to the technical tasks mentioned above. Other technical tasks not mentioned may be clearly understood by a person skilled in the art to which the present disclosure belongs from following descriptions.

### Technical Solution

A diaper for sensing urine and feces according to one embodiment of the present disclosure includes a waterproof cover, an absorbing layer located on the inner side of the waterproof cover, and a lining layer located on the inner side of the absorbing layer. The lining layer includes multiple conductive sensing lines positioned on an inner surface of the lining layer that makes contact with the human body. The conductive sensing lines are arranged separately from each other. Moreover, the electrical resistance, capacitance, and inductance between the conductive sensing lines changes upon contact between the conductive sensing lines and urine or feces.

Each of the conductive sensing lines may include a material with electrical conductivity, have the form of a thread or a band, and be formed on the lining layer using a sewing process, an adhesion process, or a printing process.

The distal end of each of the conductive sensing lines may be coupled and electrically connected to a conductive electrode-receiving portion formed on the lining layer or the waterproof cover. The distal end of each of the conductive sensing lines may be coupled to the conductive electrode-receiving portion by a sewing process using a conductive thread, a binding process using a conductive thread, an adhesion process using a conductive adhesive, or a combination thereof.

The electrode-receiving portion may include woven fabric, knitted material, felt, or non-woven material composed of conductive fibers.

One end of the lining layer on which the electrode-receiving portion is formed may be folded toward and attached to the outer surface of the waterproof cover so that the electrode-receiving portion may be exposed to the outer surface of the waterproof cover.

The electrode-receiving portion may be formed on the outer surface of the waterproof cover. One end of each of the conductive sensing lines formed on the lining layer may be coupled and electrically connected to the electrode-receiving portion through a sewing process using a conductive thread, a binding process using a conductive thread, an adhesion process using a conductive adhesive, or a combination thereof.

The conductive sensing lines may include a first sensing line and a second sensing line. The electrode-receiving portion may include a first electrode-receiving portion and a second electrode-receiving portion.

The end of the first electrode-receiving portion may be additionally fixed to the lining layer or the waterproof cover through a sewing process or an adhesion process such that a space for receiving a first electrode of the urine- and feces-sensing device may be defined between the fixed ends of the first electrode-receiving portion. The end of the second electrode-receiving portion may be additionally fixed to the lining layer or the waterproof cover through a sewing process or an adhesion process such that a space for receiving a second electrode of the urine- and feces-sensing device may be defined between the fixed ends of the second electrode-receiving portion.

The conductive sensing lines may include a first sensing line and a second sensing line. The electrode-receiving portion may include a single fourth electrode-receiving portion. The two ends of the fourth electrode-receiving portion may be respectively connected to the first and second sensing lines by a sewing process using a conductive thread, a binding process using a conductive thread, an adhesion process using a conductive adhesive, or a combination thereof. Consequently, a space for receiving an electrode of the urine- and feces-sensing device may be defined between the fixed ends of the fourth electrode-receiving portion.

The fourth electrode-receiving portion may include a first conductive cloth connected to the first sensing line, a second conductive cloth connected to the second sensing line, and a non-conductive connector connecting the first conductive cloth with the second conductive cloth separated from the first.

The conductive sensing lines may include multiple unit-sensing lines implemented with an electrical connection and spaced apart from each other, such that a urine- and feces-sensing region may be divided into sub-regions, thereby to realize efficient use of the diaper.

A diaper for sensing urine or feces according to one embodiment of the present disclosure includes a waterproof cover, an absorbing layer located on the inner side of the waterproof cover, and a lining layer located on the inner side of the absorbing layer. The lining layer includes a single conductive sensing line positioned on the inner surface of the lining layer that makes contact with the human body. Upon contact between the conductive sensing line and urine or feces, at least one of the electrical resistance, capacitance, and inductance between both ends of the conductive sensing line changes.

The conductive sensing line may be at least partially bent on the inner surface of the lining layer such that the two portions of the conductive sensing line extend parallelly and face each other.

The conductive sensing line may include a carbon fiber or a conductive polymer fiber.

A urine- and feces-sensing sheet according to one embodiment of the present disclosure is attached to the inner surface of the diaper to give the diaper a urine- and feces-sensing function. The diaper includes a waterproof cover, an absorbing layer. The urine- and feces-sensing sheet includes multiple conductive sensing lines positioned on the inner surface of the urine- and feces-sensing sheet. The inner surface of the urine- and feces-sensing sheet may be attached to the lining layer and contacted with the human body. The conductive sensing lines are arranged in a spaced manner from each other. At least one of the electrical resistance, capacitance, and inductance between the conductive sensing lines changes upon contact between the conductive sensing lines and urine or feces.

The urine- and feces-sensing sheet may be composed of the same material as the lining layer or a material with an absorbency power equal to a absorbency power of the lining layer. The urine- and feces-sensing sheet may include an attachment feature on an attachment surface that makes contact with the lining layer for user convenience.

Each of the conductive sensing lines of the sheet may include a material with electrical conductivity, have the form of a thread or a band, and be formed on the urine- and feces-sensing sheet using a sewing process, an adhesion process, or a printing process.

The distal end of each of the conductive sensing lines of the sheet may be coupled and electrically connected to a conductive electrode-receiving portion formed on the urine- and feces-sensing sheet. The coupling therebetween may be achieved through a sewing process using a conductive thread, a binding process using a conductive thread, an adhesion process using a conductive adhesive, or a combination thereof.

The electrode-receiving portion of the sheet may include woven fabric, knitted material, felt, or non-woven material composed of conductive fibers.

One end of the urine- and feces-sensing sheet on which the electrode-receiving portion is formed may be folded toward and attached to the outer surface of the waterproof cover of the diaper so that the electrode-receiving portion may be exposed to the outer surface of the waterproof cover.

The conductive sensing lines of the sheet may include a first sensing line and a second sensing line. The electrode-receiving portion may include a first electrode-receiving portion and a second electrode-receiving portion.

The end of the first electrode-receiving portion of the sheet may be fixed to the urine- and feces-sensing sheet by a sewing process or an adhesion process such that a space for receiving a first electrode of the urine- and feces-sensing device may be defined between the fixed ends of the first electrode-receiving portion. The end of the second electrode-receiving portion of the sheet may be fixed to the urine- and feces-sensing sheet by a sewing process or an adhesion process such that a space for receiving a second electrode of the urine- and feces-sensing device may be defined between the fixed ends of the second electrode-receiving portion.

The conductive sensing lines of the sheet may include a first sensing line and a second sensing line. The electrode-receiving portion may include a single fourth electrode-receiving portion. The two ends of the fourth electrode-receiving portion may be respectively connected to the first and second sensing lines through a sewing process using a conductive thread, a binding process using a conductive thread, an adhesion process using a conductive adhesive, or a combination thereof, such that a space for receiving an electrode of the urine- and feces-sensing device may be defined between the fixed ends of the fourth electrode-receiving portion.

The fourth electrode-receiving portion of the sheet may include a first conductive cloth connected to the first sensing line, a second conductive cloth connected to the second sensing line, and a non-conductive connector to connect the first conductive cloth and the second conductive cloth with each other in a spaced manner from each other.

The conductive sensing lines of the sheet may include multiple unit-sensing lines implemented with an electrical connection and spaced apart from each other, such that a urine- and feces-sensing region may be divided into sub-regions, thereby to realize efficient use of the diaper.

A urine- and feces-sensing sheet according to one embodiment of the present disclosure is attached to the inner surface of a diaper to give the diaper a urine- and feces-sensing function. The diaper includes a waterproof cover, an absorbing layer located on the inner side of the waterproof cover, and a lining layer located on the inner side of the absorbing layer. The urine- and feces-sensing sheet includes a single conductive sensing line positioned on the inner surface of the urine- and feces-sensing sheet that makes contact with the human body. The inner surface of the urine- and feces-sensing sheet is attached to the lining layer. Upon contact between the conductive sensing line and urine or feces, at least one of the electrical resistance, capacitance, and inductance between both ends of the conductive sensing line changes.

The conductive sensing line of the sheet may be at least partially bent on the inner surface of the urine- and feces-sensing sheet such that the two portions of the conductive sensing line are arranged to face each other.

The conductive sensing line of the sheet may include a carbon fiber or a conductive polymer fiber.

A urine- and feces-sensing device according to one embodiment of the present disclosure is connected to a urine- and feces-sensing diaper or to a urine- and feces-sensing sheet for a diaper. This aims to sense urine or feces based on at least one of the electrical resistance, capacitance, and inductance associated with at least one conductive sensing line positioned on the urine- and feces-sensing diaper or sheet. The device includes a urine- and feces-sensing unit for sensing absence or presence of the urine or feces of a diaper wearer based on at least one of the electrical resistance, capacitance, and inductance; a main body accommodating therein the urine- and feces-sensing unit; an electrode connected to the main body and inserted into a space defined in the electrode-receiving portion formed on the urine- and feces-sensing diaper or sheet; and a cover coupled to the main body via a rotatable hinge to pivot in a predetermined angle range for opening and closing. The cover is configured to fasten the electrode to the electrode-receiving portion.

An electrode contact may be formed on the top surface of the electrode by plating a conductive material on the electrode, ensuring that the electrode contact protrudes to a predetermined height.

The electrode contact may be electrically connected to the urine- and feces-sensing unit.

A magnet may be installed at one end of the top surface of the electrode and a magnet may be installed at one end of the bottom surface of the cover facing top surface of the electrode such that the closed state of the cover relative to the main body is maintained through attracting force between the magnets.

A spring and a soft spring cover covering the spring may be installed in a space defined on the bottom surface of the cover such that the pressing force from the spring may be maintained in the region where the electrode contact and the electrode-receiving portion overlap with each other while the electrode may be inserted into the electrode-receiving portion and the cover may be closed.

The device may further include a power switch button for powering the main body on or off.

The urine- and feces-sensing unit may wirelessly transmit urine- and feces-sensing information to a caregiver terminal or a relay communicating with the caregiver terminal.

### Advantageous Effects of the Invention

According to an embodiment of the present disclosure, the absence or presence of urine or feces of the diaper wearer may be sensed by connecting the urine- and feces-sensing device to the diaper for infants or adults equipped with the urine- and feces-sensing function.

According to an embodiment of the present disclosure, the absence or presence of urine or feces of the diaper wearer may be sensed by attaching the urine- and feces-sensing sheet to the lining of the conventional diaper for the infant or adult without a urine- and feces-sensing function, and connecting the urine- and feces-sensing device to the sheet.

According to an embodiment of the present disclosure, transmitting the urine- and feces-sensing results of the diaper wearer to the terminal of the caregiver or the repeater may allow the caregiver to change the diaper quickly.

The effects that may be obtained by the present disclosure are not limited to the effects mentioned above. Other effects not mentioned will be clearly understood by those skilled in the art to which the present disclosure belongs from the description below.

### DESCRIPTION OF THE DRAWINGS

FIG. 1 is a diagram for illustrating a configuration of a diaper.

FIG. 2 and FIG. 3 are perspective views showing inner and outer sides of a diaper for sensing urine or feces according to an embodiment of the present disclosure, respectively.

FIG. 4 is a perspective view showing a folded state of the diaper for sensing urine or feces according to an embodiment of the present disclosure.

FIG. 5 and FIG. 6 are perspective views showing inner and outer sides of a diaper for sensing urine or feces according to another embodiment of the present disclosure, respectively.

FIG. 7 is a perspective view showing a folded state of the diaper for sensing urine or feces according to another embodiment of the present disclosure.

FIG. 8 and FIG. 9 are views showing inner and outer sides of a diaper for sensing urine or feces according to another embodiment of the present disclosure, respectively.

FIG. 10 is a view showing a state in which the diaper for sensing urine or feces according to another embodiment of the present disclosure is worn on a body.

FIG. 11 and FIG. 12 are perspective views showing inner and outer sides of a diaper for sensing urine or feces according to another embodiment of the present disclosure, respectively.

FIG. 13 is a perspective view showing a folded state of the diaper for sensing urine or feces according to another embodiment of the present disclosure.

FIG. 14 to FIG. 16 are views for illustrating a modification of a conductive sensing line according to another embodiment of the present disclosure.

FIG. 17 and FIG. 18 are perspective views respectively showing inner and outer sides of a diaper for sensing urine or feces according to another embodiment of the present disclosure.

FIG. 19 is a perspective view showing a folded state of the diaper for sensing urine or feces according to another embodiment of the present disclosure.

FIG. 20 and FIG. 21 are perspective views showing inner and outer sides of a urine- and feces-sensing sheet for a diaper according to one embodiment of the present disclosure, respectively.

FIG. 22 and FIG. 23 are perspective views showing inner and outer sides of a urine- and feces-sensing sheet for a diaper according to an embodiment of the present disclosure, respectively.

FIG. 24 is a perspective view showing an outer side of a diaper attached to an urine- and feces-sensing sheet for a diaper according to an embodiment of the present disclosure.

FIG. 25 is a view showing an inner side of an urine- and feces-sensing sheet for a diaper according to another embodiment of the present disclosure.

FIG. 26 is a view showing an inner side of a urine- and feces-sensing sheet for a diaper according to another embodiment of the present disclosure.

FIG. 27 is a perspective view showing a state in which a cover of a urine- and feces-sensing device according to an embodiment of the present disclosure is closed.

FIG. 28 is a perspective view showing a state in which a cover of a urine- and feces-sensing device according to an embodiment of the present disclosure is opened.

FIG. 29 is a perspective view showing a urine- and feces-sensing device according to an embodiment of the present disclosure, as viewed from below.

FIG. 30 is a cross-sectional view showing a state in which a cover of a urine- and feces-sensing device according to an embodiment of the present disclosure is opened.

FIG. 31 is a cross-sectional view showing a state in which a cover of a urine- and feces-sensing device according to an embodiment of the present disclosure is closed.

FIG. 32 is a perspective view showing a state in which a urine- and feces-sensing device is mounted on a diaper for sensing urine or feces according to an embodiment of the present disclosure.

FIG. 33 is a perspective view showing a state in which a urine- and feces-sensing device is mounted on a diaper for sensing urine or feces according to another embodiment of the present disclosure.

FIG. 34 is a perspective view showing a state in which a urine- and feces-sensing device is mounted on a diaper for sensing urine or feces according to another embodiment of the present disclosure.

FIG. 35 is a perspective view showing a state in which a urine- and feces-sensing device is mounted on a diaper for sensing urine or feces according to another embodiment of the present disclosure.

FIG. 36 is a perspective view showing a state in which a urine- and feces-sensing device is mounted on a diaper for sensing urine or feces according to another embodiment of the present disclosure.

FIG. 37 is a perspective view showing a state in which a urine- and feces-sensing device is mounted on a urine- and feces-sensing sheet for a diaper according to an embodiment of the present disclosure as attached to a conventional diaper.

FIG. 38 is an enlarged view of an electrode-receiving portion according to another embodiment of the present disclosure.

FIG. 39 is a perspective view showing a state in which a cover of a urine- and feces-sensing device as inserted into an electrode-receiving portion according to another embodiment of the present disclosure is closed.

FIG. 40 is a perspective view showing a state in which a cover of a urine- and feces-sensing device as inserted into an electrode-receiving portion according to another embodiment of the present disclosure is opened.

FIG. 41 is a perspective view of a urine- and feces-sensing device as inserted into an electrode-receiving portion according to another embodiment of the present disclosure, as viewed from below.

FIG. 42 is a cross-sectional view showing a state in which a cover of a urine- and feces-sensing device as inserted into an electrode-receiving portion according to another embodiment of the present disclosure is opened.

FIG. 43 is a cross-sectional view showing a state in which a cover of a urine- and feces-sensing device as inserted into the electrode-receiving portion according to another embodiment of the present disclosure is closed.

FIG. 44 is a perspective view showing a state in which an electrode of a urine- and feces-sensing device is inserted into an electrode-receiving portion according to another embodiment of the present disclosure and a cover thereof is opened.

FIG. 45 is a perspective view showing a state in which an electrode of a urine- and feces-sensing device is inserted into an electrode-receiving portion according to another embodiment of the present disclosure and a cover thereof is closed.

FIG. 46 is a block diagram illustrating a schematic configuration of a diaper for sensing urine or feces and a urine- and feces-sensing device according to an embodiment of the present disclosure.

FIG. 47 is a view showing a state of sensing absence or presence of urine or feces using a urine- and feces-sensing sheet for sensing urine or feces according to an embodiment of the present disclosure.

FIG. 48 is a diagram showing an example of a resistance value measured when sensing feces, according to one embodiment of the present disclosure.

FIG. 49 is a diagram showing an example of a resistance value measured when sensing urine, in accordance with one embodiment of the present disclosure.

### BEST MODE

The advantages and/or features of the present disclosure and how to achieve them will become apparent by referring to the embodiments described below in detail in conjunction with the accompanying drawings. However, the present disclosure is not limited to the embodiments disclosed below, but will be implemented in various different forms. These embodiments are provided only to allow the present disclosure to be complete and to completely inform the person of ordinary skill in the technical field to which the present disclosure belongs of the scope of the disclosure. The present disclosure is only defined by the scope of the claims. The same reference numerals refer to the same components throughout the specification.

Hereinafter, embodiments of the present disclosure will be described in detail with reference to the accompanying drawings.

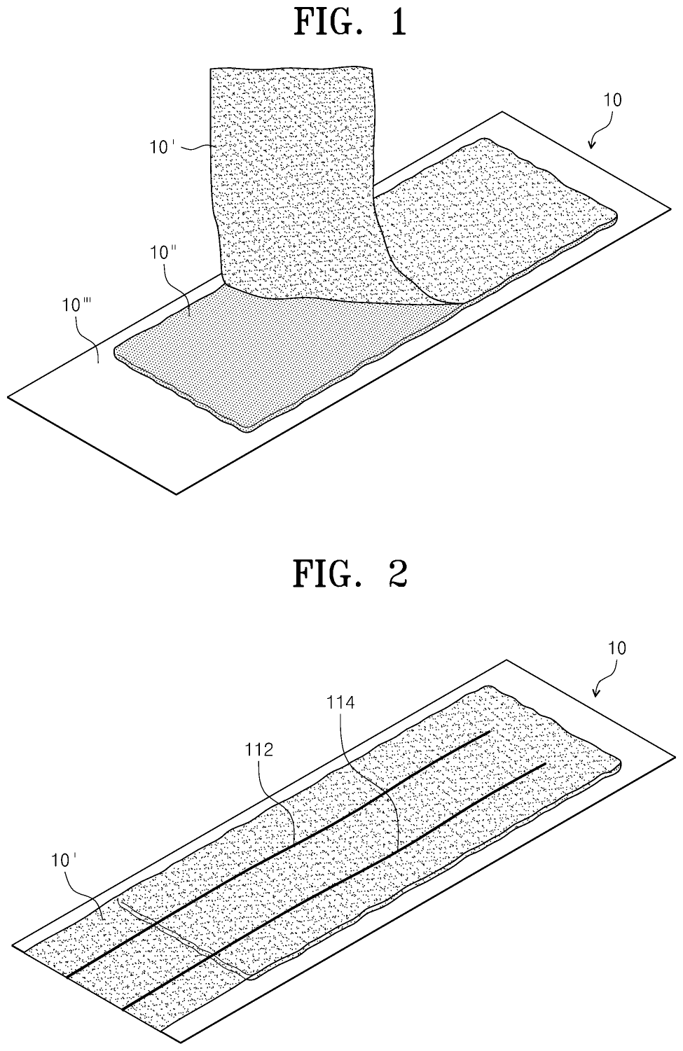

FIG. 1 is a diagram for illustrating a configuration of a diaper 10.

The diaper 10 shown in FIG. 1 may be a pad type diaper. However, the diaper 10 may be implemented as a band type diaper or a panties type diaper in addition to the pad type diaper. In addition, as long as the diaper 10 is worn on a wearer and urine or feces are collected by the diaper 10, the type of the diaper is not limited thereto. For reference, the diaper wearer may be applied to animals as well as human as adults or children.

Referring to FIG. 1, the diaper 10 may have a structure in which a lining layer 10′, an absorbing layer 10″, and a waterproof cover 10′″ are sequentially stacked vertically. The lining layer 10′ is located on the inner side of the absorbing layer 10″, and the absorbing layer 10″ is located on the inner side of the waterproof cover 10′″.

The lining layer 10′ is a layer through which urine or feces are permeable. The lining layer 10′ may be divided into an inner surface that directly contacts the diaper wearer's skin and an outer surface that contacts the diaper or a member capable of receiving urine or feces.

According to one embodiment of the present disclosure, the lining layer 10′ may be embodied using a fabric including cloth, knitted fabric, felt, or nonwoven material, but may not be limited thereto, and may be embodied using various materials.

The absorbing layer 10″ is located on the outer surface of the lining layer 10′ to absorb and receive urine or feces delivered through the lining layer 10′.

The waterproof cover 10″ is located on the outer surface of the absorbing layer 10″ to prevent urine or feces absorbed in the absorbing layer 10″ from leaking out of the diaper 10.

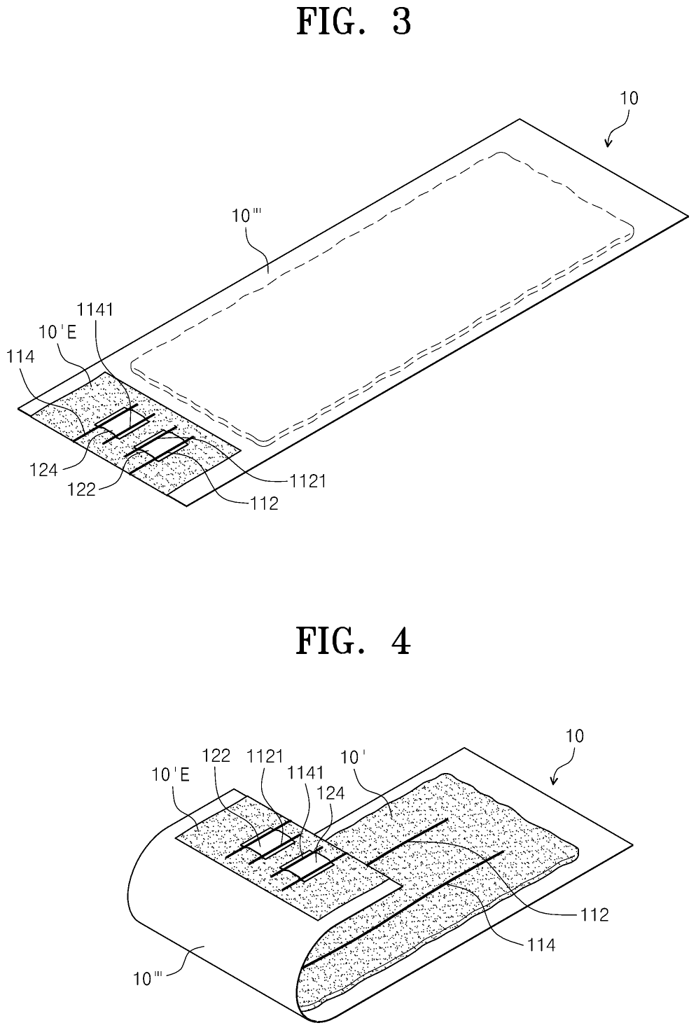

FIG. 2 and FIG. 3 are perspective views showing inner and outer sides of the diaper 10 for sensing urine or feces according to one embodiment of the present disclosure, respectively. FIG. 4 is a perspective view showing a folded state of the diaper 10 for sensing urine or feces according to an embodiment of the present disclosure.

Referring to FIG. 2 to FIG. 4, the diaper 10 for sensing the urine or feces includes the waterproof cover 10′″, the absorbing layer 10″ located on the inner side of the waterproof cover 10′″, and the lining layer 10′ located on the inner side of the absorbing layer 10″.

According to an embodiment of the present disclosure, the lining layer 10′ includes multiple conductive sensing lines 112 and 114 that are positioned on the inner surface thereof that contacts a human body, and are arranged separately from each other. Upon contact between the conductive sensing lines 112 and 114 and urine or feces, at least one of the electrical resistance, capacitance, and inductance between the conductive sensing lines 112 and 114 may change.

According to an embodiment of the present disclosure, each of the conductive sensing lines 112 and 114 may include a material with electrical conductivity. According to this embodiment, each of the conductive sensing lines 112 and 114 may be composed of a material with electrical conductivity while being harmless to the human body or less toxic to the skin. For example, the conductive material included in each of the conductive sensing lines 112 and 114 may be silver. The conductive material is not limited to silver. Other conductive materials such as gold, stainless steel, copper alloy (brass, white copper, etc.), titanium, aluminum, etc. may be included in addition to the silver.

According to an embodiment of the present disclosure, the conductive sensing lines 112 and 114 may be formed on the lining layer 10′ through a sewing process, an adhesion process, or a printing process.

Further, the diaper 10 for sensing the urine or feces includes conductive electrode-receiving portions 122 and 124.

Referring to FIG. 3 and FIG. 4, the electrode-receiving portions 122 and 124 may be formed on a portion of the lining layer 10′. However, the disclosure is not limited thereto. The electrode-receiving portions 122 and 124 may be formed on the waterproof cover 10′″.

According to an embodiment of the present disclosure, the distal end of each of the conductive sensing lines 112 and 114 is electrically connected to each of the electrode-receiving portions 122 and 124 to form an electrical unity. For example, as shown in FIG. 3 and FIG. 4, each of the conductive sensing lines 112 and 114 and each of the electrode-receiving portions 122 and 124 may be connected to each other through a sewing process using a conductive thread, a binding process using a conductive thread or an adhesion process using a conductive adhesive. In addition, each of the conductive sensing lines 112 and 114 and each of the electrode-receiving portions 122 and 124 may be connected to each other through a combination of the processes as described above.

Each of the electrode-receiving portions 122 and 124 may be composed of a fabric, knitted fabric, felt or non-woven material composed of conductive fibers and thus have electrical conductivity.

According to an embodiment of the present disclosure, the conductive sensing lines 112 and 114 and the electrode-receiving portions 122 and 124 connected thereto respectively may be formed on the lining layer 10′. One end of the lining layer 10′ may be folded toward the outer surface of the waterproof cover 10′″ and attached to the waterproof cover 10′″.

Specifically, referring to FIG. 3 and FIG. 4, the conductive sensing lines 112 and 114 and the electrode-receiving portions 122 and 124 may be formed on an inner surface of the lining layer 10′. A portion 10′E of the lining layer 10′ on which the electrode-receiving portions 122 and 124 are formed may extend beyond the waterproof cover 10′″. The portion 10′E may be folded toward the outer surface of the waterproof cover 10′″. And then the portion 10′E may be attached to the waterproof cover 10′″ with a tape or adhesive.

As a result, the electrode-receiving portions 122 and 124 may be exposed to the outer surface of the waterproof cover 10′″ and may be combined with the urine- and feces-sensing device as described later.

In this way, the electrode-receiving portions 122 and 124 are formed on the outer surface of the waterproof cover 10′″. One end of each of the conductive sensing lines 112 and 114 formed on the lining layer 10′ is electrically connected to each of the electrode-receiving portions 122 and 124. As described above, each of the electrode-receiving portions 122 and 124 and each of the conductive sensing lines 112 and 114 may be electrically coupled to each other through a sewing process using a conductive thread, a binding process using a conductive thread, and an adhesion process using a conductive adhesive or through a combination of the processes.

As described above, in the diaper 10 for sensing urine or feces as shown in FIG. 2 to FIG. 4, the conductive sensing lines 112 and 114 include the first sensing line 112 and the second sensing line 114. Moreover, the electrode-receiving portions 122 and 124 include the first electrode-receiving portion 122 and the second electrode-receiving portion 124.

In this connection, as shown in FIG. 3 and FIG. 4, one side of the first electrode-receiving portion 122 (the outer side of the diaper in FIG. 3 and FIG. 4) is coupled to the first sensing line 112 and is fixed to the lining layer 10′. The other side of the first electrode-receiving portion 122 (the inner side of the diaper in FIG. 3 and FIG. 4) is additionally fixed to the lining layer 10′ through a sewing process or an adhesion process.

Similarly, one side of the second electrode-receiving portion 124 (the outer side of the diaper in FIG. 3 and FIG. 4) is coupled to the second sensing line 114 and fixed to the lining layer 10′. The other side of the second electrode-receiving portion 124 (the inner side of the diaper in FIG. 3 and FIG. 4) is additionally fixed to the lining layer 10′ through a sewing process or an adhesion process.

As a result, a space in which each electrode of the urine- and feces-sensing device as described later may be inserted is defined between one side and the other side of each of the first and second electrode-receiving portions 122 and 124.

In this connection, when the other side of each of the first and second electrode-receiving portions 122 and 124 (that is, the inner side of the diaper) is fixed to the lining layer 10′ through a sewing process, the other side of each of the first and second electrode-receiving portions 122 and 124 may be fixed thereto using each of first and second fixing lines 1121 and 1141.

In this case, the first and second fixing lines 1121 and 1141 may be composed of general non-conductive threads, unlike the first and second sensing lines 112 and 114 with electrical conductivity. When the first and second fixing lines 1121 and 1141 are composed of an electrically conductive thread, the first and second fixing lines 1121 and 1141 are connected to each other due to fluffing thereof. In such cases, the first and second electrode-receiving portions 122 and 124 are shorted with each other, and thus there is a possibility that an abnormality occurs in the urine- and feces-sensing function.

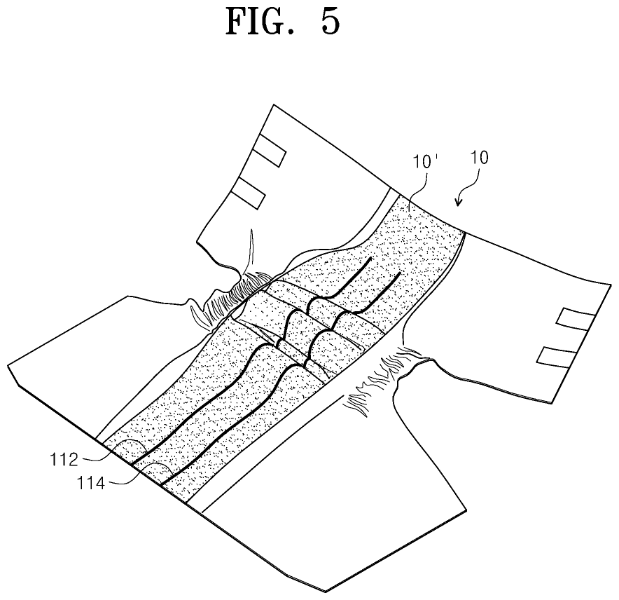

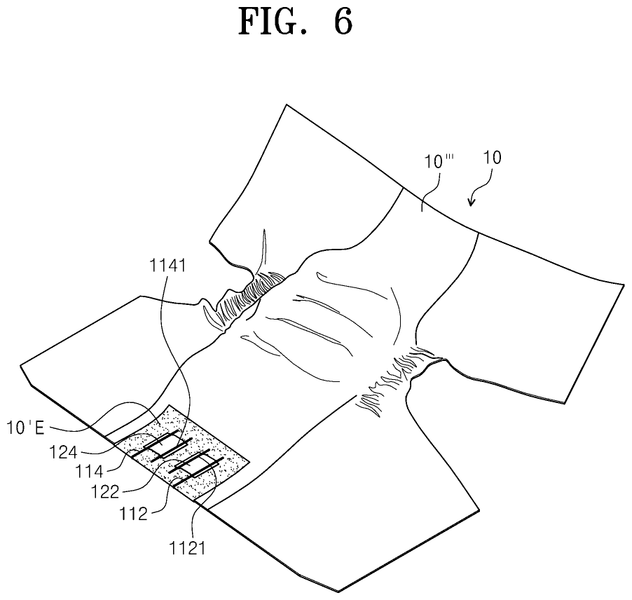

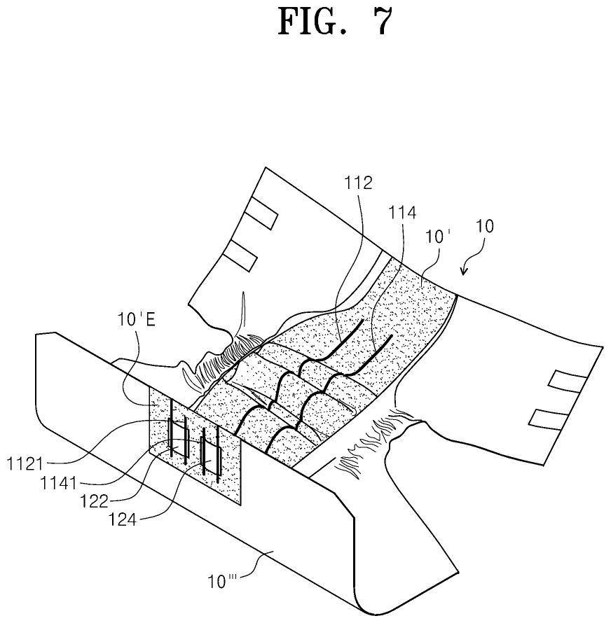

FIG. 5 and FIG. 6 are perspective views showing inner and outer sides of a diaper 10 for sensing urine or feces according to another embodiment of the present disclosure, respectively. FIG. 7 is a perspective view showing a folded state of the diaper 10 for sensing urine or feces according to another embodiment of the present disclosure.

The diaper 10 for sensing urine or feces as described above is implemented as a pad type diaper, whereas in this embodiment, the diaper is implemented as a band type diaper. Referring to FIG. 5 to FIG. 7, even in this embodiment, the band type diaper 10 with the urine- and feces-sensing function includes the waterproof cover 10′″, the absorbing layer 10″, and the lining layer 10′. The lining layer 10′ includes multiple conductive sensing lines 112 and 114. At least one of the electrical resistance, capacitance, and inductance between the conductive sensing lines 112 and 114 changes upon contact with urine or feces.

In addition, referring to FIG. 6 and FIG. 7, the band type diaper 10 having the urine- and feces-sensing function has the conductive sensing lines 112 and 114 and the electrode-receiving portions 122 and 124 formed on the inner surface of the lining layer 10′. The portion 10′E of the lining layer 10′ on which each of the electrode-receiving portions 122 and 124 is formed may extend beyond the waterproof cover 10″. The portion 10′E may be folded toward the outer surface of the waterproof cover 10′″. And the portion 10′E may be attached to the waterproof cover 10′″ with a tape or adhesive.

As a result, also in this embodiment, the electrode-receiving portions 122 and 124 may be exposed to the outer surface of the waterproof cover 10′″ and combined with the urine- and feces-sensing device as described later.

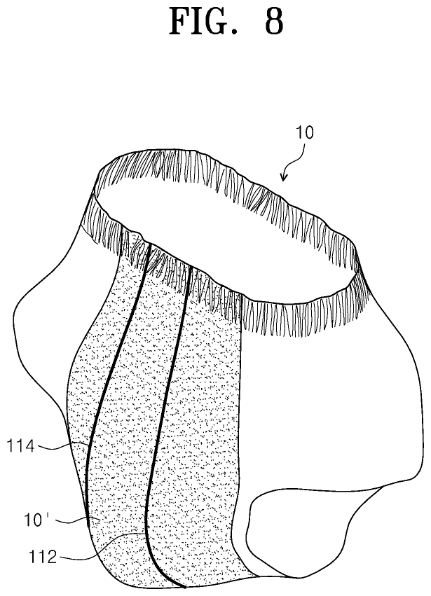

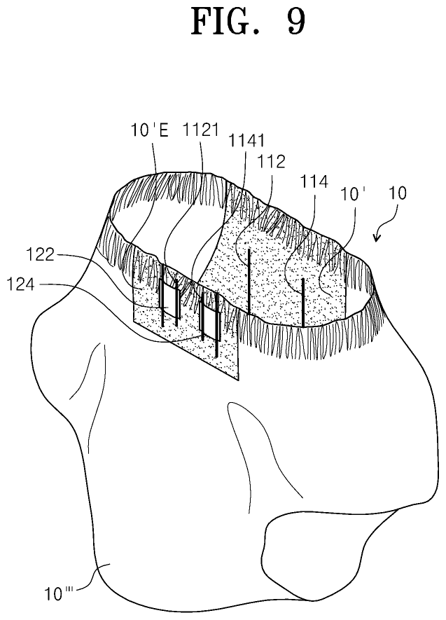

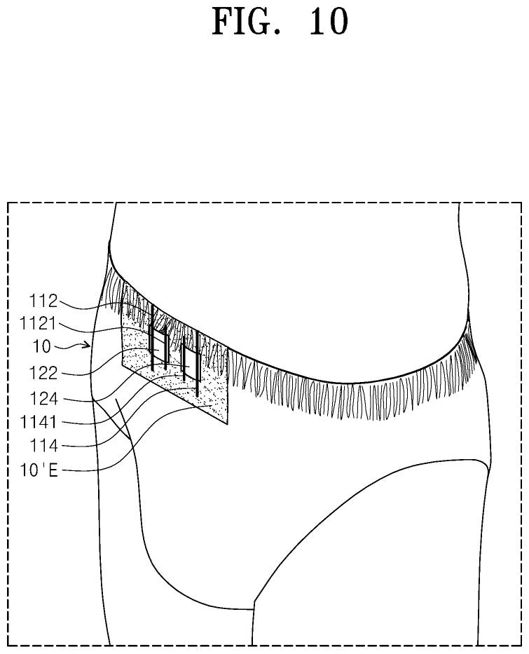

FIG. 8 and FIG. 9 are diagrams showing inner and outer sides of a diaper 10 for sensing urine or feces according to another embodiment of the present disclosure, respectively. FIG. 10 is a diagram showing a state in which the diaper 10 for sensing urine or feces according to another embodiment of the present disclosure is worn on a body.

Unlike the embodiments as described above, in this embodiment, the diaper 10 for sensing urine and feces is embodied as a panties type. Referring to FIG. 8 to FIG. 10, similarly, in this embodiment, the panties type diaper 10 having urine- and feces-sensing function includes the waterproof cover 10″, the absorbing layer 10″, and the lining layer 10′. The lining layer 10′ includes the conductive sensing lines 112 and 114. At least one of the electrical resistance, capacitance, and inductance between the conductive sensing lines 112 and 114 changes upon contact with urine or feces.

In addition, referring to FIG. 9 and FIG. 10, the panties type diaper 10 having the urine- and feces-sensing function has the conductive sensing lines 112 and 114 and the electrode-receiving portions 122 and 124 formed on the inner surface of the lining layer 10′. The portion 10′E of the lining layer 10′ on which each of the electrode-receiving portions 122 and 124 is formed may extend beyond the waterproof cover 10″. The portion 10′E may be folded toward the outer surface of the waterproof cover 10′″. And the portion 10′E may be attached to the waterproof cover 10′″ with a tape or adhesive.

As a result, also in this embodiment, the electrode-receiving portions 122 and 124 may be exposed to the outer surface of the waterproof cover 10′″ and combined with the urine- and feces-sensing device as described later.

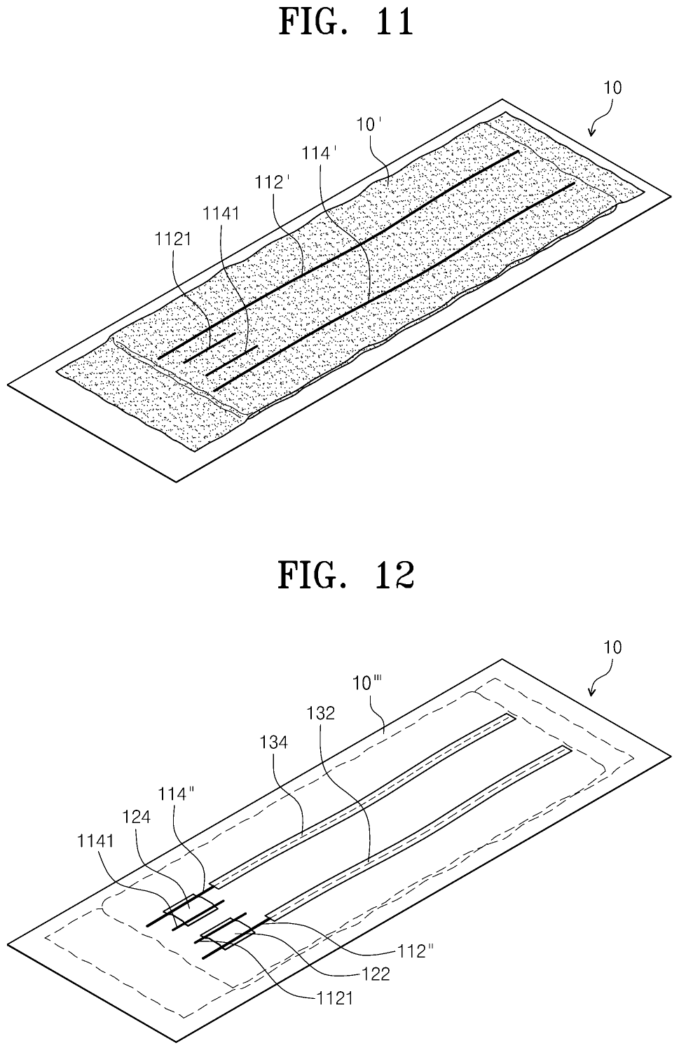

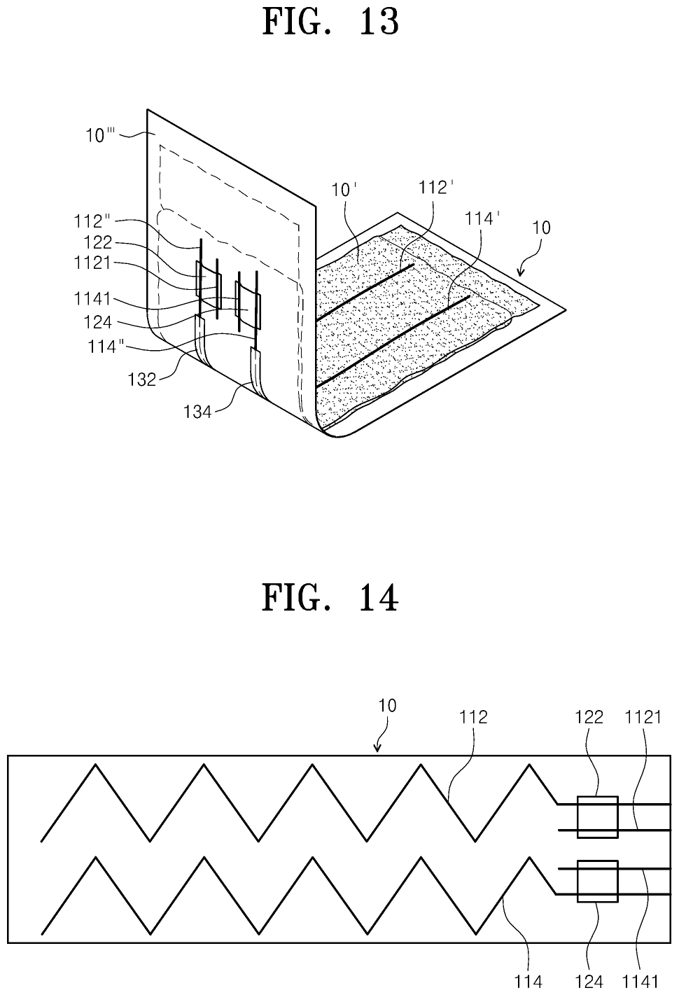

FIG. 11 and FIG. 12 are perspective views showing inner and outer sides of a diaper 10 for sensing urine or feces according to another embodiment of the present disclosure, respectively. FIG. 13 is a perspective view showing a folded state of the diaper 10 for sensing urine or feces according to another embodiment of the present disclosure.

According to this embodiment, in the diaper 10 for sensing the urine or feces, the conductive sensing lines may be mounted on each of the inner side and the outer side of the diaper 10 through a sewing process.

Specifically, referring to FIG. 11 to FIG. 13, the diaper 10 for sensing the urine or feces may include a first conductive sensing line 112′ and a second conductive sensing line 114′ formed on the inner surface of the lining layer 10′, and a third conductive sensing line 112″ and a fourth conductive sensing line 114″ formed on the outer surface of the waterproof cover 10′″.

The first and second conductive sensing lines 112′ and 114′ are electrically connected to the third and fourth conductive sensing lines 112″ and 114′, respectively.

In this embodiment, the first conductive sensing line 112′ and the third conductive sensing line 112″ may be implemented as a lower-thread and a upper-thread in the sewing process, respectively. The second conductive sensing line 114′ and the fourth conductive sensing line 114″ may be implemented as a lower-thread and an upper-thread in the sewing process, respectively.

According to this embodiment, in the diaper 10 for sensing the urine or feces, each of the first and second sensing lines 112′ and 114′ and each of the third and fourth sensing lines 112″ and 114″ may extend from the inner surface of the lining layer 10′ though the absorbing layer 10″ to the outer surface of the waterproof cover 10′″ and may be connected to each other, thus enabling more reliable urine- and feces-sensing.

In addition, the first and second conductive sensing lines 112′ and 114′ formed on the body side of the diaper wearer in the diaper 10 are buried in the lining layer 10′ through the sewing process, such that the conductive sensing lines do not directly contact the skin, and, thus, trouble on the skin may not occur.

Furthermore, according to this embodiment, adding a process of sewing the conductive sensing lines on the conventional diaper may allow the diaper 10 for sensing urine or feces according to the present disclosure to be manufactured without significant change to a conventional diaper production line.

Depending on an embodiment, the third sensing line 112″ formed on the outer surface of the waterproof cover 10′″ may be integral with the first sensing line 112′ formed on the inner surface of the lining layer 10′. Similarly, the fourth sensing line 114″ formed on the outer surface of the waterproof cover 10′″ may be integral with the second sensing line 114′ formed on the inner surface of the lining layer 10′. That is, in this case, the conductive sensing lines are not sewn as a combination of the lower-thread and upper-threads, but may be sewn into one thread.

Further, as shown in FIG. 12 and FIG. 13, the diaper 10 for sensing the urine or feces may further include waterproof tapes 132 and 134, respectively on at least a portion of the third sensing line 112″ and at least a portion of the fourth sensing line 114″ and on the outer surface of the waterproof cover 10′″ to block leakage of urine or feces through a sewing mark.

In one example, the conductive sensing lines may be formed in various patterns.

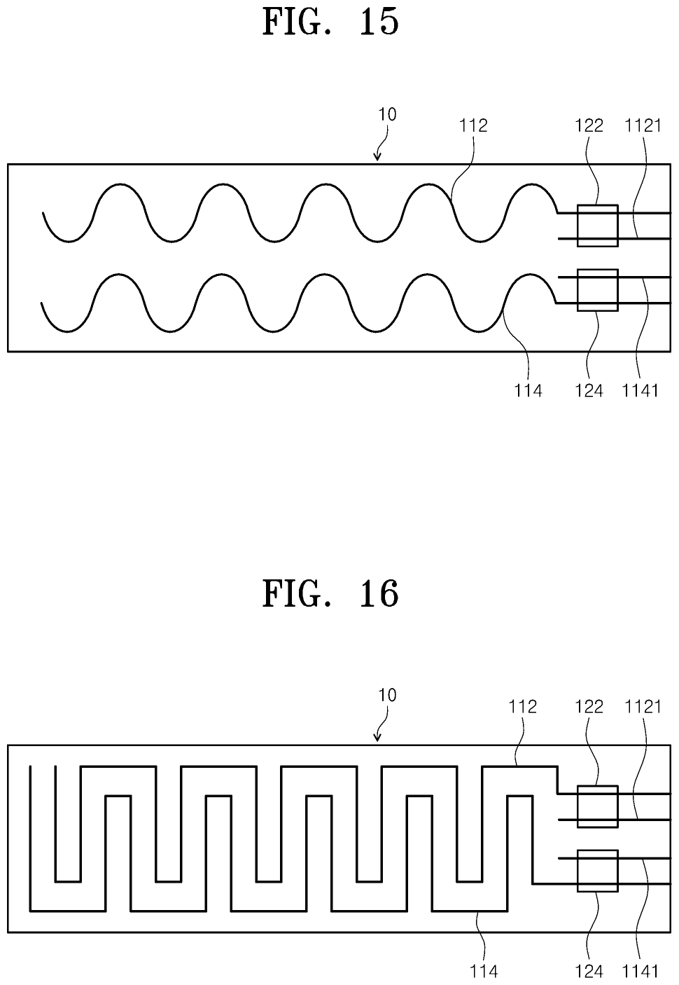

For example, as shown in FIG. 14, the conductive sensing lines 112 and 114 may be formed in a zigzag pattern. As shown in FIG. 15, the conductive sensing lines 112 and 114 may be formed in a wave pattern along a length direction. As shown in FIG. 16, the conductive sensing lines 112 and 114 may be formed in a pulse pattern along the length direction.

For reference, in the present embodiment, the sensing lines for sensing the urine or feces are arranged in two rows, but is not limited thereto, and may be implemented to have three or more rows.

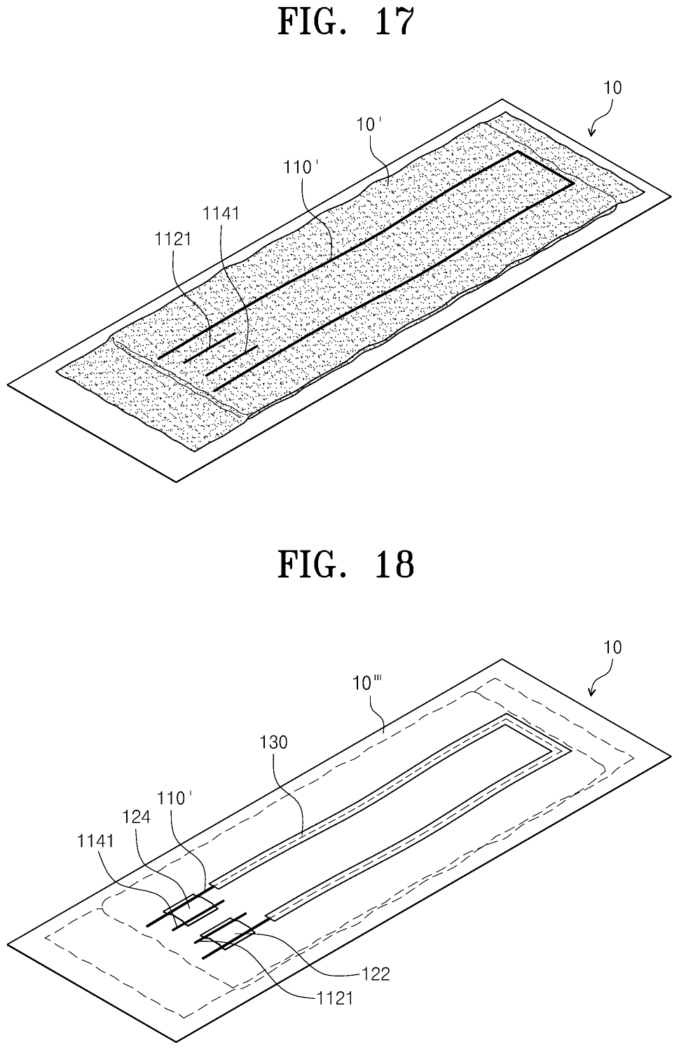

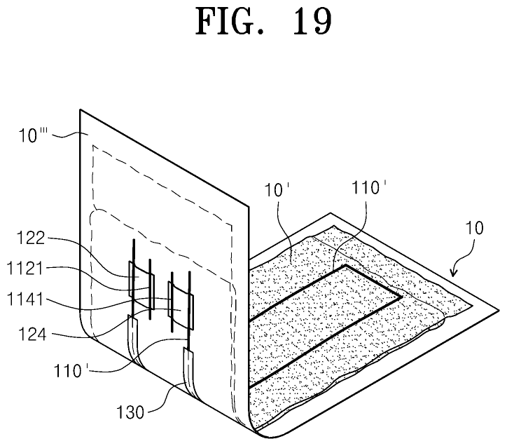

FIG. 17 and FIG. 18 are perspective views showing inner and outer sides of a diaper 10 for sensing urine or feces according to another embodiment of the present disclosure, respectively. FIG. 19 is a perspective view showing a folded state of the diaper 10 for sensing urine or feces according to another embodiment of the present disclosure.

Whereas in the above-described embodiment, the conductive sensing lines 112 and 114 are arranged separately from each other on the lining layer 10′ of the diaper 10, according to this embodiment, the diaper 10 includes a single conductive sensing line 110′.

Referring to FIG. 17, in the diaper 10 for sensing urine or feces according to another embodiment of the present disclosure, one portion of the single conductive sensing line 110′ and the other portion thereof may extend parallelly each other. For example, the single conductive sensing line 110′ may be positioned on the lining layer 10′ so that a middle portion of the single conductive sensing line 110′ is bent at a right angle so that one portion of the single conductive sensing line 110′ and the other portion thereof extend parallelly and face each other.

Each of both ends of the conductive sensing line 110′ may be electrically coupled to one side (outer side in FIG. 18) of each of the electrode-receiving portions 122 and 124. Moreover, the other side (inner side in FIG. 18) of each of the electrode-receiving portions 122 and 124 may be fixed to the diaper 10. Thus, a space into which an electrode of the urine- and feces-sensing device described later is inserted may be defined between the fixed sides of the electrode-receiving portions 122 and 124.

Specifically, when the conductive sensing lines 112 and 114 described above are used, each of the conductive sensing lines 112 and 114 may include a high conductive material, and thus may have as low electrical resistance as possible. Thus, electrical resistance between the conductive sensing lines 112 and 114 is usually at a very high state. However, when urine or feces are present between the conductive sensing lines 112 and 114, the electrical resistance between the conductive sensing lines 112 and 114 is lowered, so that current flows well therebetween.

To the contrary, when the single conductive sensing line 110′ is used as in this embodiment, the single conductive sensing line 110′ may include a material having a low conductivity and have as high electrical resistance as possible. In this case, usually, the electrical resistance of the conductive sensing line 110′ is higher than a certain level. However, when urine and feces are present between the opposing portions of the conductive sensing line 110′, current flows through the urine and feces having a higher conductivity than that of the conductive sensing line 110′. Thus, the electrical resistance measured across both ends of the conductive sensing line 110′ is lower than that measured usually.

Therefore, in the embodiment as shown in FIG. 17 to FIG. 19, the single conductive sensing line 110′ may include a conductive material whose conductivity is lower than that of general urine or feces.

In one example, the conductive material of each of the conductive sensing lines 112 and 114 as described above includes silver or other conductive materials. In this embodiment, the conductive material of the conductive sensing line 110′ may include a carbon fiber or a conductive polymer fiber.

The single conductive sensing line 110′ instead of the conductive sensing lines 112 and 114 may be equally applied to the lining layer 10′ of each of the pad type, band type and panties type diapers 10 shown in FIGS. 1 to 10.

Further, as shown in FIG. 18, when the single conductive sensing line 110′ is formed on the diaper 10 through the sewing process, the diaper 10 for sensing the urine or feces may further include a waterproof tape 130 on at least a portion of the conductive sensing line 110′ and on the outer surface of the waterproof cover 10′″, thereby to block leakage of urine or feces through a sewing mark.

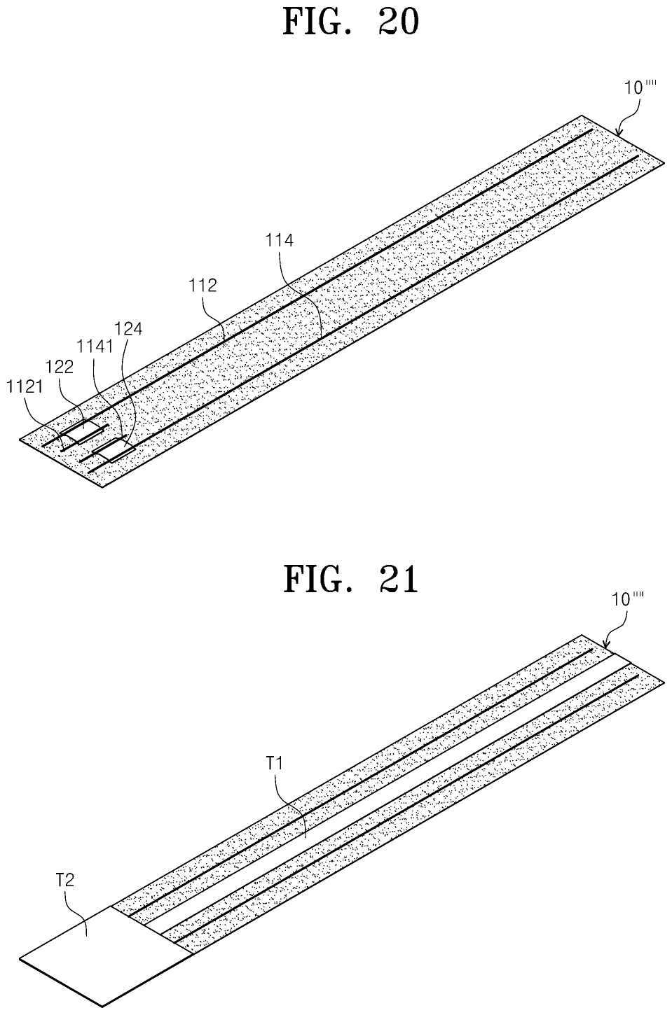

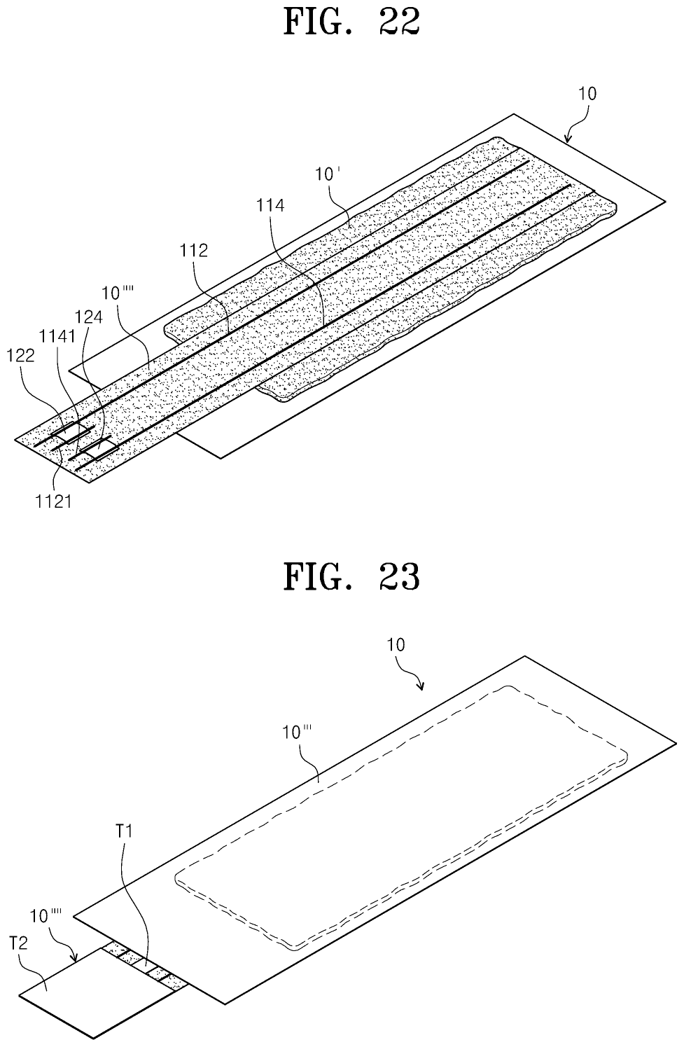

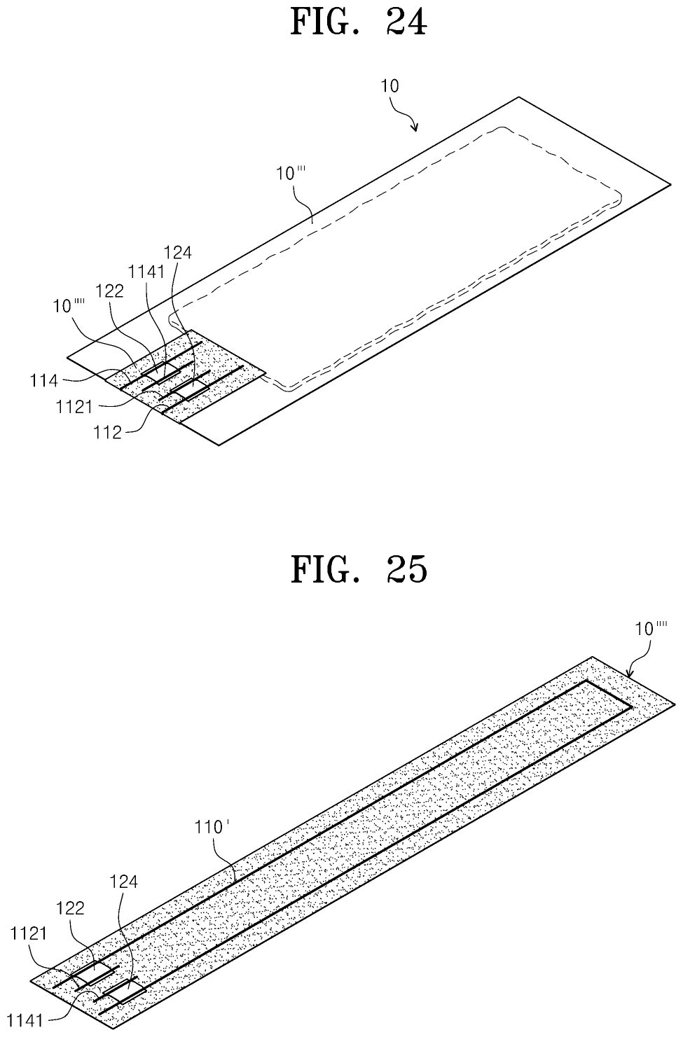

FIG. 20 and FIG. 21 are perspective views showing inner and outer sides of a urine- and feces-sensing sheet 10″ for a diaper according to one embodiment of the present disclosure, respectively. FIG. 22 and FIG. 23 are perspective views showing inner and outer sides of a diaper 10 on which the urine- and feces-sensing sheet 10″ for the diaper according to an embodiment of the present disclosure is positioned, respectively. FIG. 24 is a perspective view showing an outer side of a diaper 10 having the urine- and feces-sensing sheet 10″ for the diaper according to an embodiment of the present disclosure attached thereto.

The urine- and feces-sensing sheet 10″″ according to an embodiment of the present disclosure is a sheet that may be attached to an inner side of a conventional diaper to give the conventional diaper the urine- and feces-sensing function.

The urine- and feces-sensing sheet 10″″ is attached to the lining layer of the conventional diaper 10 and contacts a human body. Moreover, as shown in FIG. 20, the urine- and feces-sensing sheet 10″ includes multiple conductive sensing lines 112 and 114 arranged separately from each other and positioned on the inner surface thereof.

At least one of the electrical resistance, capacitance, and inductance between the conductive sensing lines 112 and 114 may change upon contact between the conductive sensing lines 112 and 114 and urine or feces.

The urine- and feces-sensing sheet 10″ may be composed of the same material as that of the lining layer 10′ of the diaper 10, or a material with absorbency power corresponding to that of the lining layer 10′.

Moreover, the urine- and feces-sensing sheet 10″″ may include a predetermined attachment feature on an attachment surface thereof that makes contact with the lining layer 10′ and is attached to the lining layer 10′. For example, as shown in FIG. 21, a tape T1 to be attached to the lining layer 10′ of the diaper 10 may be positioned on the outer surface of the urine- and feces-sensing sheet 10″.

A configuration of the conductive sensing lines 112 and 114 provided in the urine- and feces-sensing sheet 10″ according to an embodiment of the present disclosure is the same as the configuration of the conductive sensing lines 112 and 114 provided in the diaper 10 for sensing urine or feces according to the embodiments of the present disclosure as described above.

Furthermore, the urine- and feces-sensing sheet 10″ includes multiple electrode-receiving portions 122 and 124. The electrode-receiving portions 122 and 124 are respectively connected to the conductive sensing lines 112 and 114 in an electrically integrated manner. A configuration of the electrode-receiving portions 122 and 124 is the same as the configuration of the conductive sensing lines 112 and 114 provided in the diaper 10 for sensing urine or feces according to the embodiments of the present disclosure as described above.

Further, referring to FIG. 22 and FIG. 23, in the urine- and feces-sensing sheet 10″, like the lining layer 10′ of the diaper 10 for sensing urine or feces according to embodiments of the present disclosure as described above, a portion on which each of the electrode-receiving portions 122 and 124 is formed extends beyond the waterproof cover 10′″ of the diaper 10. And, as shown in FIG. 24, the extending portion is folded toward the outer surface of the waterproof cover 10′″ and attached to the diaper using the predetermined attachment feature.

To this end, as shown in FIG. 21, a tape T2 may be provided on the outer surface of the urine- and feces-sensing sheet 10″″ so that the urine- and feces-sensing sheet may be attached to the waterproof cover 10′″.

As a result, the electrode-receiving portions 122 and 124 formed on the urine- and feces-sensing sheet 10″ may be exposed to the outer surface of the waterproof cover 10′″ of the diaper 10.

FIG. 25 is a view showing an inner side of a urine- and feces-sensing sheet 10″ for a diaper according to another embodiment of the present disclosure.

In the embodiment as described above, the conductive sensing lines 112 and 114 are arranged separately on the urine- and feces-sensing sheet 10″″. However, according to this embodiment, the urine- and feces-sensing sheet 10″″ includes the single conductive sensing line 110′.

Referring to FIG. 25, the urine- and feces-sensing sheet 10″ according to another embodiment of the present disclosure. One portion of the single conductive sensing line 110′ and the other portion thereof may extend in a parallel manner and face each other. For example, the single conductive sensing line 110′ may be positioned on the urine- and feces-sensing sheet 10″″ so that a middle portion of the single conductive sensing line 110′ is bent at a right angle so that one portion of the single conductive sensing line 110′ and the other portion thereof extend parallelly and face each other.

Moreover, each of both ends of the conductive sensing line 110′ may be electrically coupled to one side (outer side in FIG. 25) of each of the electrode-receiving portions 122 and 124. Moreover, the other side (inner side in FIG. 25) of each of the electrode-receiving portions 122 and 124 may be fixed to the urine- and feces-sensing sheet 10″″. Thus, a space into which an electrode of the urine- and feces-sensing device as described later is inserted may be defined between the fixed sides of the electrode-receiving portions 122 and 124.

A configuration of the single conductive sensing line 110′ in the urine- and feces-sensing sheet 10″″ according to this embodiment, and an effect thereof may be the same as those of the single conductive sensing line 110′ included in the diaper 10 for sensing the urine or feces as described above.

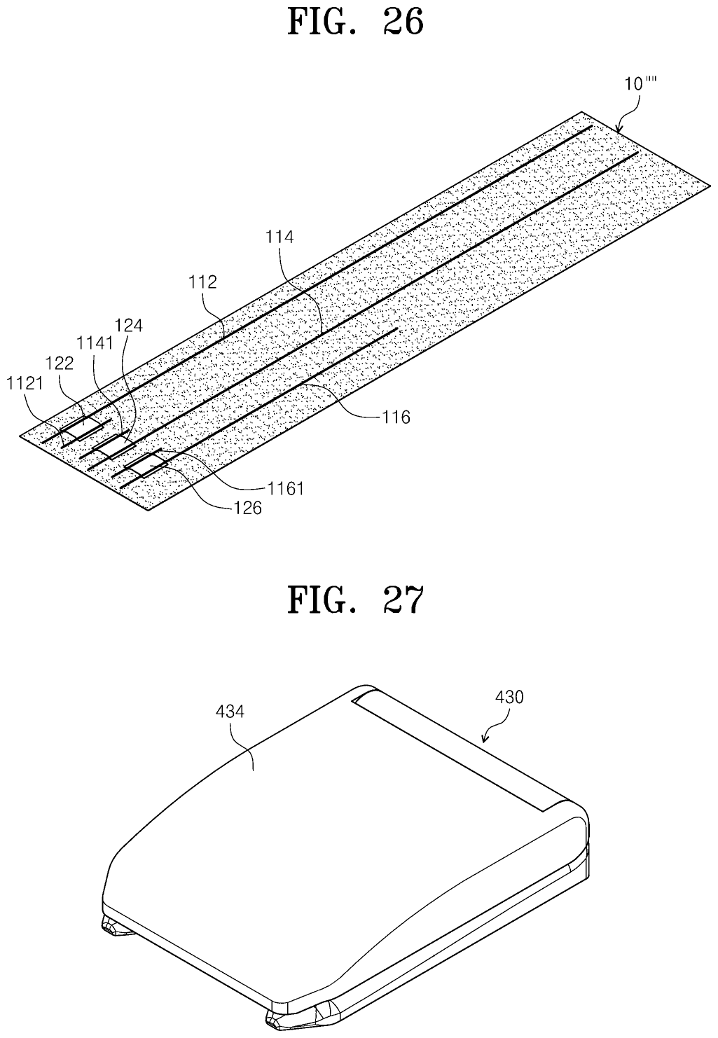

FIG. 26 is a view showing an inner side of a urine- and feces-sensing sheet 10″ for a diaper according to another embodiment of the present disclosure.

In the previous embodiment, when the urine- and feces-sensing sheet 10″″ has the conductive sensing lines, the conductive sensing lines include the two conductive sensing lines, that is, the first and second conductive sensing lines 112 and 114.

However, depending on an embodiment, the urine- and feces-sensing sheet 10″ may include three or more conductive sensing lines. Furthermore, when the urine- and feces-sensing sheet 10″ includes three or more conductive sensing lines, the conductive sensing lines may not have the same length. Thus, lengths of some of the three or more conductive sensing lines may be different from those of the rest thereof, or lengths of the three or more conductive sensing lines may be different from each other.

For example, referring to FIG. 26, the urine- and feces-sensing sheet 10″″ may include the first conductive sensing line 112, the second conductive sensing line 114 and a third conductive sensing line 116. Moreover, the urine- and feces-sensing sheet 10″ may include the first electrode-receiving portion 122 electrically connected to the first conductive sensing line 112, the second electrode-receiving portion 124 electrically connected to the second conductive sensing line 114, and a third electrode-receiving portion 126 electrically connected to the third conductive sensing line 116.

One side of each of the first to third electrode-receiving portions 122 124, and 126 may be fixed to the urine- and feces-sensing sheet 10″″ through each of the first to third conductive sensing lines 112, 114, and 116. The other side of each of the first to third electrode-receiving portions 122 124, and 126 may be fixed to the urine- and feces-sensing sheet 10″ through each of first to third fixing lines 1121, 1141, and 1161. In this case, each of the first to third fixing lines 1121, 1141, and 1161 may be composed of a general thread that does not have conductivity, unlike the first to third conductive sensing lines 112, 114, and 116.

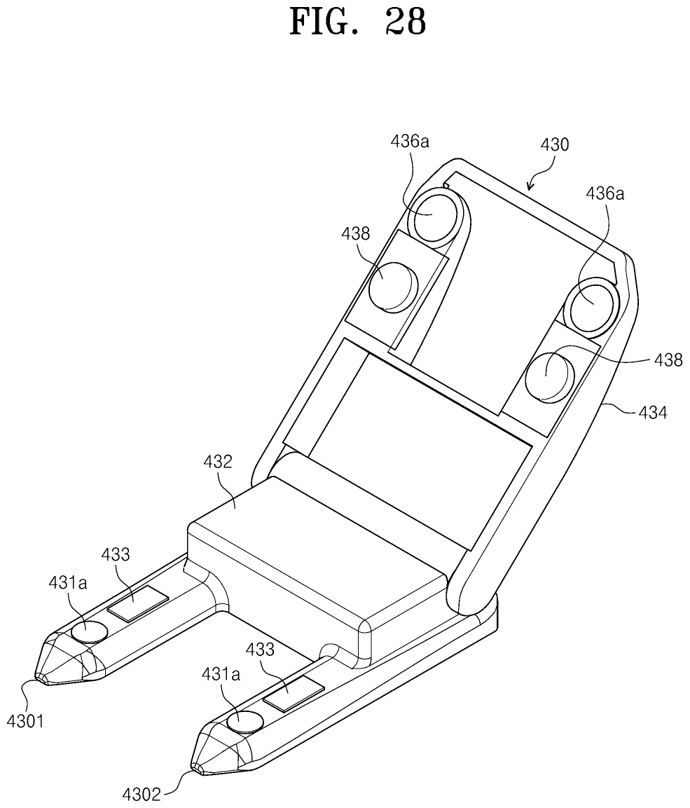

When the urine- and feces-sensing sheet 10″ as shown in FIG. 26 is applied to the diaper 10, the urine- and feces-sensing device as described with reference to FIG. 27 to FIG. 31 may include the number of electrodes corresponding to the number of the electrode-receiving portions. In other words, as described below, the urine- and feces-sensing device coupled to the electrode-receiving portions of the urine- and feces-sensing sheet 10″ to detect presence or absence of urine or feces may include the number of electrodes corresponding to the number of the electrodes receiving portions 122, 124, and 126 provided in the urine- and feces-sensing sheet 10″.

According to this embodiment, as shown in FIG. 26, one of the first to third conductive sensing lines 112, 114, and 116 provided in the urine- and feces-sensing sheet 10″″, for example, the third conductive sensing line 116 may be shorter than the other two, that is, the first and second conductive sensing lines 112 and 114.

In FIG. 26, the third conductive sensing line 116 having the shortest length may be positioned at the outermost side of the urine- and feces-sensing sheet 10″″. However, depending on an embodiment, the shortest conductive sensing line may be positioned in a middle of the urine- and feces-sensing sheet 10″. That is, the third conductive sensing line 116 having the shortest length may be positioned between the first and second conductive sensing lines 112 and 114 having a longer length.

When the lengths of the three or more conductive sensing lines 112, 114, and 116 provided in the urine- and feces-sensing sheet 10″″ are different from each other, the urine and feces may be individually sensed in each of different regions of the urine- and feces-sensing sheet 10″″, for example, in an anterior region corresponding to a lower abdomen and a posterior region corresponding to a buttocks of the diaper wearer.

The configuration of the three or more conductive sensing lines described above is not limited to being applied to the urine- and feces-sensing sheet 10″″ but may also be applied to the lining layer 10′ of each of the diapers 10 of various types as mentioned above.

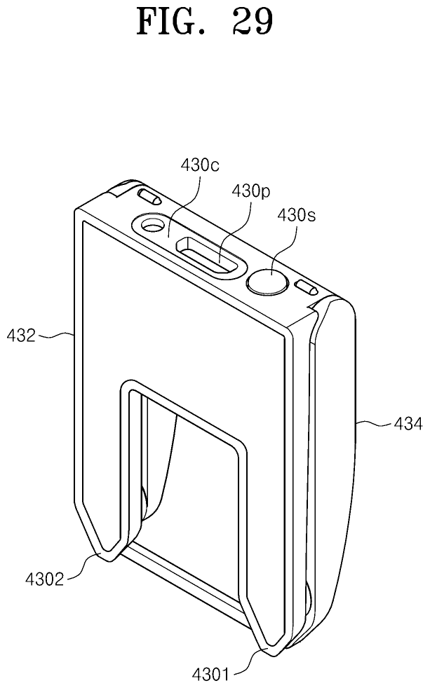

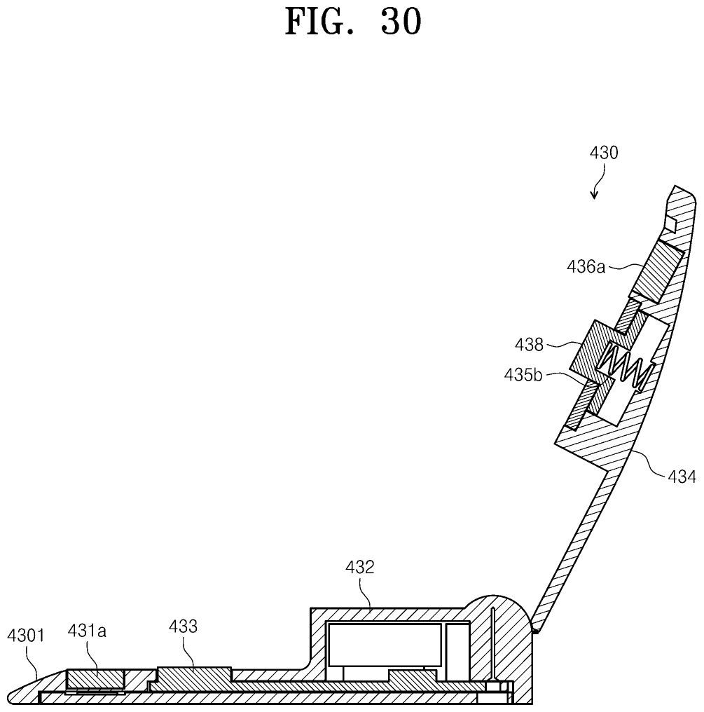

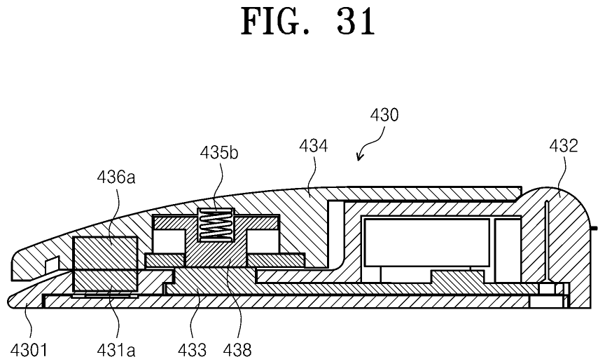

FIG. 27 is a perspective view showing a state in which a cover of a urine- and feces-sensing device 430 according to an embodiment of the present disclosure is closed. FIG. 28 is a perspective view showing a state in which the cover of the urine- and feces-sensing device 430 according to an embodiment of the present disclosure is opened. FIG. 29 is a perspective view showing the urine- and feces-sensing device 430 according to an embodiment of the present disclosure as viewed from below. FIG. 30 is a cross-sectional view showing a state in which the cover of urine- and feces-sensing device 430 according to an embodiment of the present disclosure is opened. FIG. 31 is a cross-sectional view showing a state in which the cover of the urine- and feces-sensing device 430 according to an embodiment of the present disclosure is closed.

The urine- and feces-sensing device 430 according to one embodiment of the present disclosure is connected to the diaper 10 for sensing the urine or feces as described above or the urine- and feces-sensing sheet 10″ for the diaper as described above to sense the urine or feces.

Referring to FIG. 27 to FIG. 31, the urine- and feces-sensing device 430 includes a urine- and feces-sensing unit (not shown), a main body 432, electrodes 4301 and 4302, and a cover 434.

The urine- and feces-sensing unit may be configured to sense absence or presence of urine or feces of the diaper wearer, based on at least one of the electrical resistance, capacitance, and inductance between the conductive sensing lines 112 and 114 or of the single conductive sensing line 110′.

For example, the urine- and feces-sensing unit measures electrical resistance between both ends of the conductive sensing lines. When the measured electrical resistance becomes smaller than a preset value, the urine- and feces-sensing unit may determine that the diaper 10 has urine or feces.

The main body 432 accommodates the urine- and feces-sensing unit therein.

The electrodes 4301 and 4302 may protrude from one end of the main body 432, and may be inserted respectively into the spaces defined in the electrode-receiving portions 122 and 124 formed on the diaper 10 or the urine- and feces-sensing sheet 10″″.

The electrodes 4301 and 4302 protruding from one end of the urine- and feces-sensing device 430 may be inserted respectively into the spaces defined in the electrode-receiving portions 122 and 124 formed on the lining layer 10′ of the diaper 10 or the urine- and feces-sensing sheet 10″″. Thus, the urine- and feces-sensing device 430 may be coupled to the diaper 10.

The cover 434 may be coupled to the main body 432 through a rotatable hinge for opening and closing while pivoting in a predetermined angle range. When the cover 434 is closed, the electrodes 4301 and 4302 and the electrode-receiving portions 122 and 124 may be in close contact with each other.

Referring to FIG. 28 and FIG. 30, on the top surface of each of the electrodes 4301 and 4302, an electrode contact 433 protruding to a predetermined height may be formed. The contact may be formed by plating a conductive material.

The electrode contact 433 may be electrically connected to the urine- and feces-sensing unit to electrically connect each of the electrode-receiving portions 122 and 124 and the urine- and feces-sensing unit with each other.

Further, as shown in FIG. 28, FIG. 30 and FIG. 31, in the urine- and feces-sensing device 430, a magnet may be installed on one end of the top surface of each of the electrode 4301 and 4302. Also a magnet may be installed on one end of the bottom surface of the cover 434 facing the top surface of each of the electrode 4301 and 4302. In FIG. 28, FIG. 30 and FIG. 31, the magnet installed on each of the electrodes 4301 and 4302 is indicated by a reference numeral 431a, while the magnet installed on the cover 434 is indicated by a reference numeral 436a.

As a result, the closed state of the cover 434 relative to the main body 432 may be maintained through a certain attracting force. In the urine- and feces-sensing device 430 shown in the drawing, the magnets are installed on both each of the electrodes 4301 and 4302, and the cover 434. However, depending on an embodiment, the magnet may be installed only on one of each of the electrodes 4301 and 4302, and the cover 434, and iron attracted by the magnet may be installed on the other.

Further, referring to FIG. 28, FIG. 30 and FIG. 31, a plunger 438 and a plunger spring 435b may be installed in a space defined on the bottom surface of the cover 434. That is, the cover 434 of the urine- and feces-sensing device 430 according to an embodiment of the present disclosure is provided with predetermined pressing means to apply a certain pressure to the electrode contact 433 while the cover 434 is closed.

As a result, when the electrodes 4301 and 4302 are inserted into the electrode-receiving portions 122 and 124 and the cover 434 is in the closed state, the certain pressing force is maintained at a portion where the electrode contacts 433 and the electrode-receiving portions 122 and 124 overlap each other.

In addition, referring to FIG. 29, the urine- and feces-sensing device 430 may further include a charging port 430P, and a charging port cover 430C in the main body 432. The urine- and feces-sensing device 430 may receive power through charging means (e.g., an HDMI cable) inserted in the charging port 430P to charge a battery (not shown) provided therein.

Further, referring to FIG. 29, the urine- and feces-sensing device 430 may further include a power switch button 430S to power on or off the main body 432. The user may turn the urine- and feces-sensing device 430 on or off by pressing the power switch button 430S.

Further, the urine- and feces-sensing unit of the urine- and feces-sensing device 430 may include a wireless communication device to wirelessly transmit urine- and feces-sensing information to a terminal of a caregiver of the diaper wearer or a relay communicating with the caregiver terminal.

For example, when the urine- and feces-sensing unit detects that the electrical resistance value between both ends of the conductive sensing lines 112, 114, and 110′ is lower than a preset reference value, the urine- and feces-sensing unit may send a RF signal to the terminal or the relay through the wireless communication device.

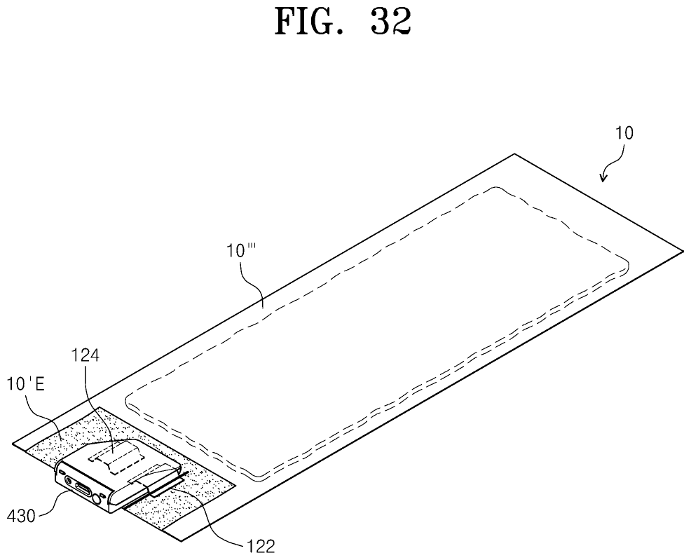

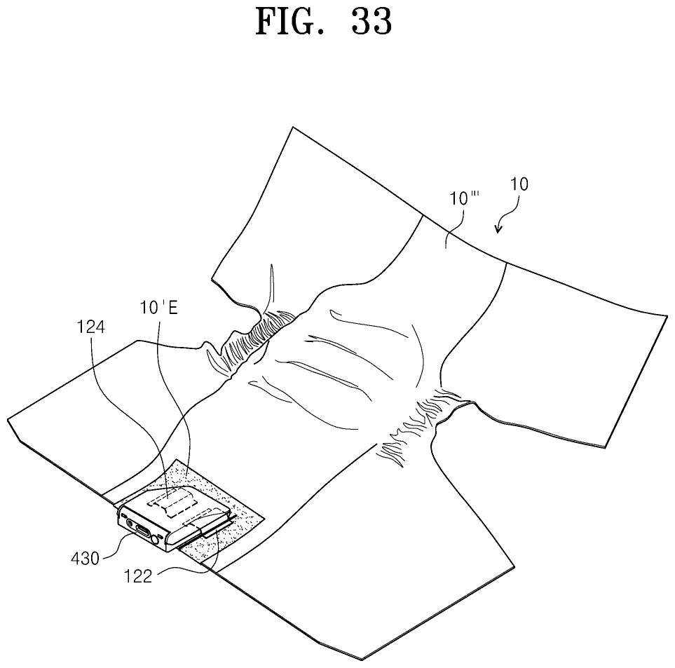

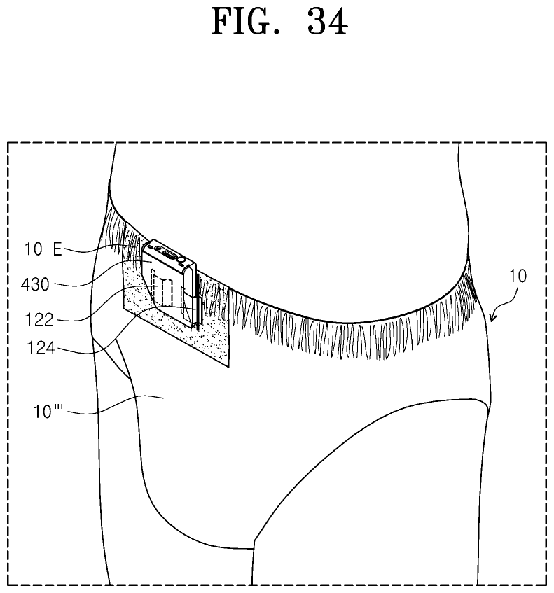

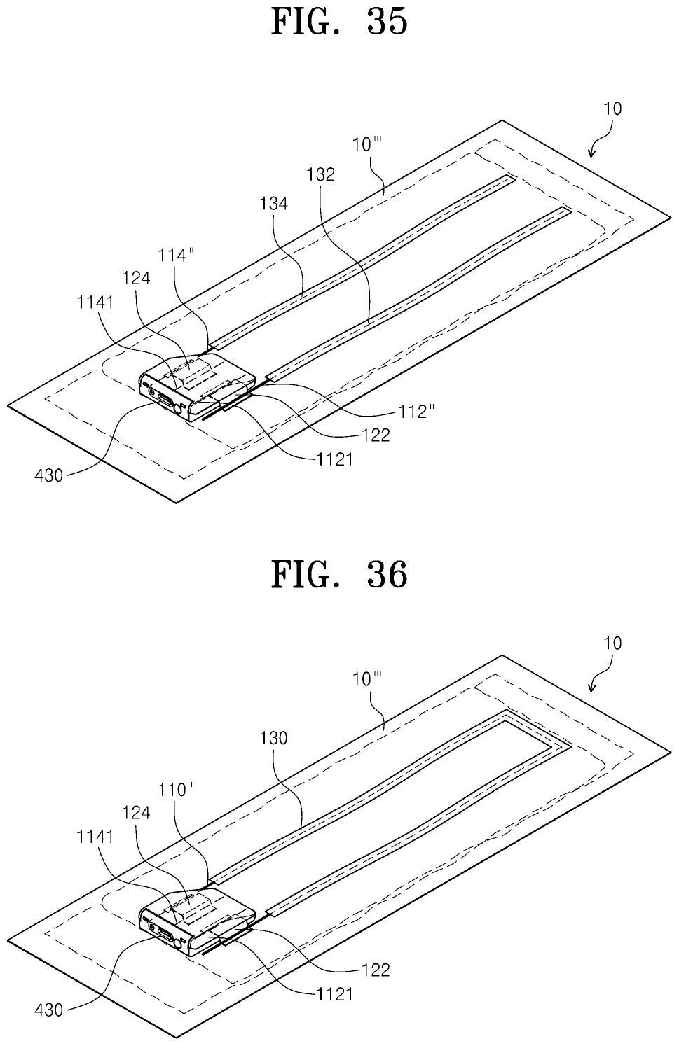

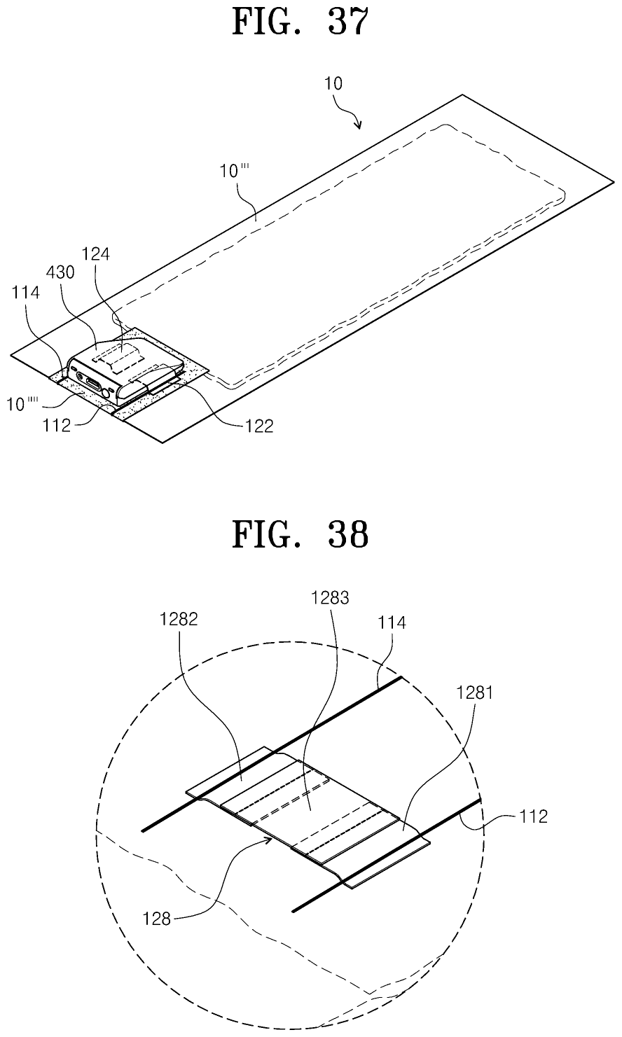

FIG. 32 is a perspective view showing a state in which the urine- and feces-sensing device 430 is mounted on the pad type diaper 10 having an urine- and feces-sensing function according to an embodiment of the present disclosure. FIG. 33 is a perspective view showing a state in which the urine- and feces-sensing device 430 is mounted on the band type diaper 10 having urine- and feces-sensing function according to another embodiment of the present disclosure. FIG. 34 is a perspective view showing a state in which the urine- and feces-sensing device 430 is mounted on the diaper 10 for sensing urine or feces according to another embodiment of the present disclosure. FIG. 35 is a perspective view showing a state in which the urine- and feces-sensing device 430 is mounted on the diaper 10 for sensing urine or feces according to another embodiment of the present disclosure. FIG. 36 is a perspective view showing a state in which the urine- and feces-sensing device 430 is mounted on the diaper 10 having the single conductive sensing line according to another embodiment of the present disclosure. FIG. 37 is a perspective view showing a state in which the urine- and feces-sensing device 430 is mounted on the urine- and feces-sensing sheet 10″ for the diaper according to an embodiment of the present disclosure attached to the diaper 10.

As shown in FIG. 32 to FIG. 37, the electrodes 4301 and 4302 of the urine- and feces-sensing device 430 may be respectively inserted into the electrode-receiving portions 122 and 124 formed on the lining layer 10′ or the urine- and feces-sensing sheet 10″, and the cover 434 may be closed. Thus, the urine- and feces-sensing device 430 may be stably coupled to the diaper 10.

The urine- and feces-sensing device 430 coupled to the outer surface of the diaper 10 may be easily detached from the diaper by the diaper wearer or the caregiver thereof.

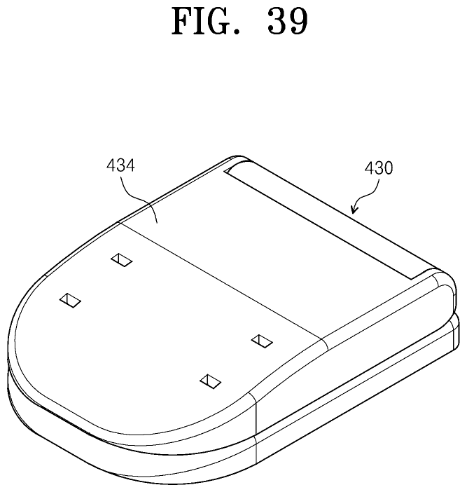

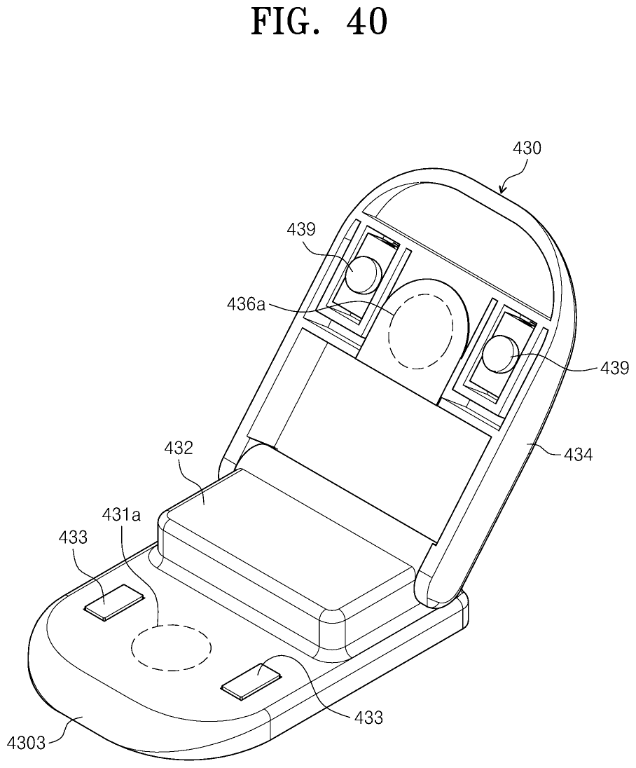

FIG. 38 is an enlarged view of an electrode-receiving portion according to another embodiment of the present disclosure.

Referring to FIG. 38, the electrode-receiving portion according to another embodiment of the present disclosure may be composed of a single fourth electrode-receiving portion 128. In other words, in this embodiment, the electrode-receiving portion may be composed only of the single fourth electrode-receiving portion 128.

According to this embodiment, the two ends of the fourth electrode-receiving portion 128 may be coupled to the first and second sensing lines 112 and 114, respectively. As a result, a space in which the electrodes of the urine- and feces-sensing device 430 are inserted may be defined between fixed ends of the fourth electrode-receiving portion 128. The coupling between the fourth electrode-receiving portion 128 and the first and second sensing lines 112 and 114 may be achieved through a sewing process using a conductive thread, a binding process using a conductive thread, an adhesion process using a conductive adhesive, or a combination of these processes.

According to this embodiment, as shown in FIG. 38, the fourth electrode-receiving portion 128 may include a first conductive cloth 1281, a second conductive cloth 1282, and a non-conductive connector 1283.

The first conductive cloth 1281 may be connected to the first sensing line 112. The second conductive cloth 1282 may be connected to the second sensing line 114. In this connection, the first conductive cloth 1281 and the second conductive cloth 1282 are separated from each other to allow an electrically non-conductive state therebetween.

Moreover, the non-conductive connector 1283 is configured to connect the first conductive cloth 1281 and the second conductive cloth 1282 separated from each other to each other in a spaced apart state therebetween. For example, as shown in FIG. 38, one side of the non-conductive connector 1283 may be coupled to the first conductive cloth 1281 through sewing, while the other side thereof may be coupled to the second conductive cloth 1282 through sewing.

The non-conductive connector 1283 is composed of an insulating material in which electricity does not flow.

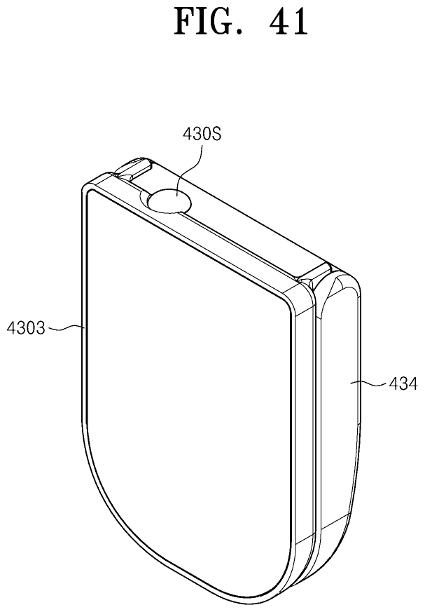

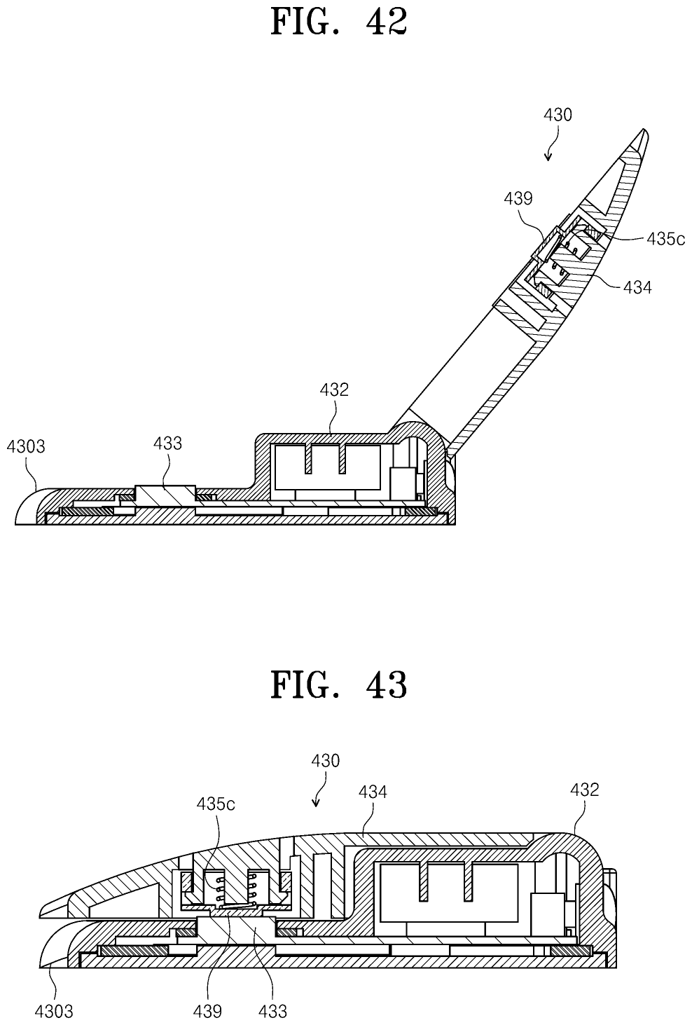

FIG. 39 is a perspective view showing a state in which the cover 434 of the urine- and feces-sensing device 430 as inserted into the electrode-receiving portion 128 according to another embodiment of the present disclosure is closed. FIG. 40 is a perspective view showing a state in which the cover 434 of the urine- and feces-sensing device 430 as inserted into the electrode-receiving portion 128 according to another embodiment of the present disclosure is opened. FIG. 41 is a perspective view showing the urine- and feces-sensing device 430 inserted in the electrode-receiving portion 128 according to another embodiment of the present disclosure as viewed from below. FIG. 42 is a cross-sectional view showing a state in which the cover 434 of the urine- and feces-sensing device 430 as inserted in the electrode-receiving portion 128 according to another embodiment of the present disclosure is opened. FIG. 43 is a cross-sectional view showing a state in which the cover 434 of the urine- and feces-sensing device 430 as inserted into the electrode-receiving portion 128 according to another embodiment of the present disclosure is closed.

Unlike the urine- and feces-sensing device 430 according to the embodiment of the present disclosure as described with reference to FIG. 27 to FIG. 31, the urine- and feces-sensing device 430 according to another embodiment of the present disclosure has a single electrode 4303.

The electrode 4303 may be connected to the main body 432, and may be inserted into a space provided in a single electrode-receiving portion 128 as shown in FIG. 38, formed on the diaper or the urine- and feces-sensing sheet.

In addition, the configurations of the urine- and feces-sensing unit (not shown), the main body 432 accommodating the urine- and feces-sensing unit therein, and the cover 434 covering the main body 432 in the urine- and feces-sensing device 430 may have the same configurations as those of the urine- and feces-sensing device according to the embodiment of the present disclosure as described above.

However, in this embodiment, since only one electrode 4303 is provided in the urine- and feces-sensing device 430, a single magnet 431a may be installed on the electrode 4303 and a single magnet 436a may be installed on the cover 434, as shown in FIG. 40.

Further, as shown in FIG. 41, instead of including the charging port and the charging port cover, the urine- and feces-sensing device 430 may include a wireless charging module for wireless charging of the battery in the main body 432 to charge the battery wirelessly.

Further, referring to FIG. 42 and FIG. 43, in the urine- and feces-sensing device 430, a spring 435c and a spring cover 439 covering the spring 435c may be installed in a space defined on the bottom surface of the cover 434.

As a result, when the electrode 4303 is inserted into the electrode-receiving portion 128 and the cover 434 is closed, the pressing force from the spring 435c may be maintained in the region where the electrode contact 433 of the urine- and feces-sensing device 430 and the electrode-receiving portion 128 overlap each other.

In this embodiment, the spring cover 439 may be composed of a soft material such as silicon. The material of the spring cover 439 is not limited thereto.

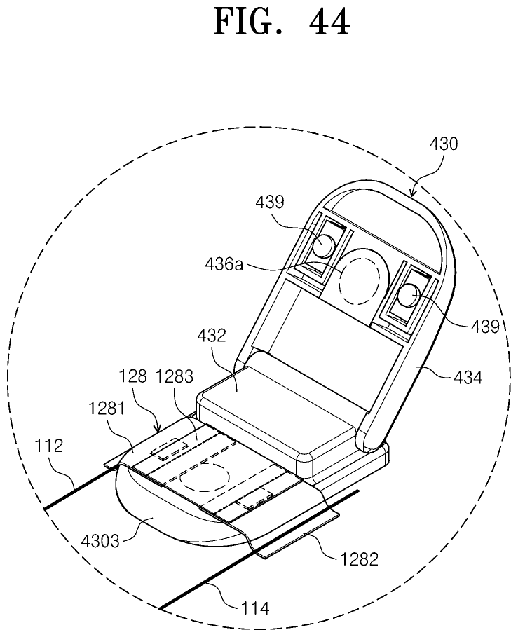

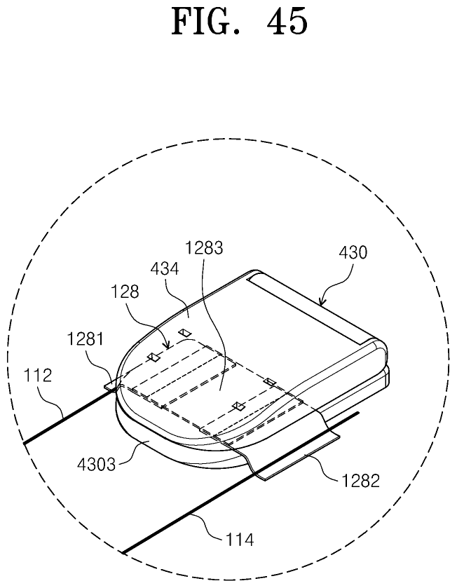

FIG. 44 is a perspective view showing a state in which the electrode 4303 of the urine- and feces-sensing device 430 is inserted in the electrode-receiving portion 128 according to another embodiment of the present disclosure and the cover 434 is opened. FIG. 45 is a perspective view showing a state in which the electrode 4303 of the urine- and feces-sensing device 430 is inserted into the electrode-receiving portion 128 according to another embodiment of the present disclosure and the cover 434 is closed.

As shown in FIG. 44, in the urine- and feces-sensing device 430 according to another embodiment of the present disclosure, a single electrode 4303 is inserted into a space defined in the single fourth electrode-receiving portion 128 such that the two electrode contacts 433 of the urine- and feces-sensing device 430 contact the first and second conductive clothes 1281 and 1282 of the fourth electrode-receiving portion 128 respectively to electrically connect the first and second sensing lines 112 and 114 with each other.

Moreover, as shown in FIG. 45, when the cover 434 of the urine- and feces-sensing device 430 is closed, the spring 435c provided in the cover 434 may apply a certain pressure to the fourth electrode-receiving portion 128 through the spring cover 439 to allow adhesion between the electrode contacts 433 of the urine- and feces-sensing device 430 and the fourth electrode-receiving portion 128 to be maintained at a certain level.

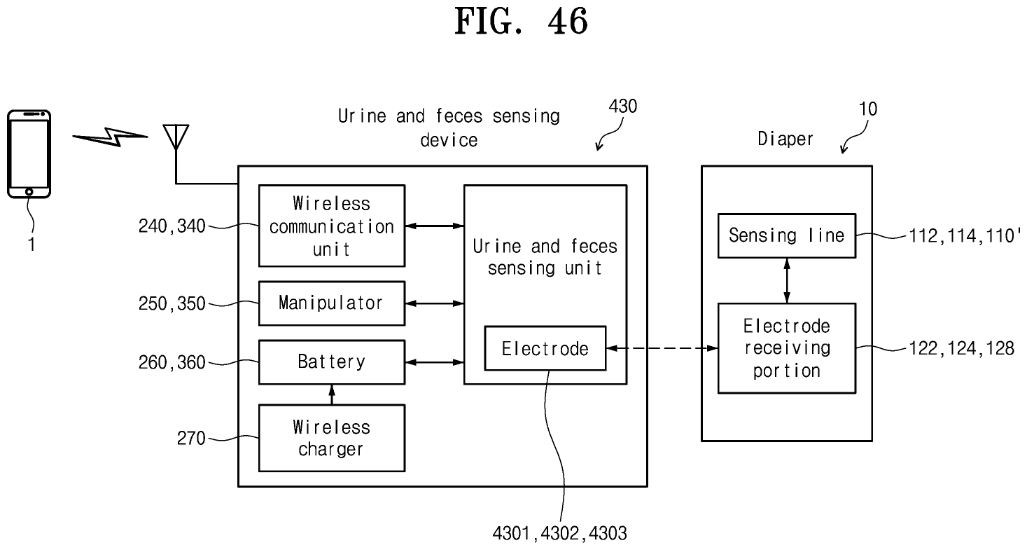

FIG. 46 is a block diagram for illustrating a schematic configuration of the diaper 10 for sensing urine or feces and the urine- and feces-sensing device 430 according to an embodiment of the present disclosure.

Referring to FIG. 46, the diaper 10 for sensing urine or feces according to an embodiment of the present disclosure includes the conductive sensing lines 112 and 114 or the single conductive sensing line 110′, and the electrode-receiving portions 122 and 124 electrically coupled to the conductive sensing line(s).

Moreover, the urine- and feces-sensing device 430 according to the embodiment of the present disclosure includes the urine- and feces-sensing unit, the electrodes 4301 and 4302, a wireless communication unit 240 or 340 and a battery 260 or 360.

The electrodes 4301 and 4302 of the urine- and feces-sensing device 430 are electrically connected to the electrode-receiving portions 122 and 124 of the diaper 10 respectively to measure at least one of the electrical resistance, capacitance, and inductance between the conductive sensing lines.

When the diaper wearer has urine or feces, the urine or feces may be present on the lining layer 10′ of the diaper 10 or the urine- and feces-sensing sheet 10″″. Thus, the urine- and feces-sensing unit may detect at least one change of electrical resistance, capacitance, and inductance between the ends of the conductive sensing lines 112 and 114 or 100′.

Moreover, when change in at least one of the electrical resistance, capacitance, and inductance between the ends of the conductive sensing lines 112, 114, and 100′ is sensed, the urine- and feces-sensing device 430 may inform, to a terminal 1 of a caregiver, that urine and feces are present on the diaper 10, through the wireless communication unit 240 or 340.

Specifically, referring to FIG. 48, when it is sensed that the diaper wearer has urine or feces, the urine- and feces-sensing unit creates and outputs urine and feces information. The output urine and feces information is transmitted to the caregiver terminal 1 through the wireless communication unit 240 or 340.

The urine- and feces-sensing unit may continuously transmit the urine and feces information at a certain period until the urine- and feces-sensing unit has received a confirmation signal (ACK signal) from the caregiver terminal 1 through the wireless communication unit 240 or 340. In this connection, the caregiver terminal 1 is not limited to a specific implementation scheme as long as the terminal 1 is configured to be capable of communicating with the wireless communication unit 240 or 340. For example, in embodiments of the present disclosure, the caregiver terminal 1 may be implemented as a smartphone equipped with a Bluetooth module.

The urine- and feces-sensing unit measures remaining power in the battery 260 or 360 and transmits the remaining power information together with the urine and feces information to the caregiver terminal 1 through the wireless communication unit 240 or 340. Alternatively, the urine- and feces-sensing unit may transmit only the remaining power information to the caregiver terminal 1 through the wireless communication unit 240 or 340 independently and at a certain time period.

The wireless communication unit 240 or 340 may be implemented using a short-range wireless communication module such as a Bluetooth communication module or a ZigBee communication module, and perform communication with the caregiver terminal 1 having a communication module corresponding thereto. For reference, the wireless communication unit 240 or 340 may use various short-range wireless communication interfaces such as Wi-Fi, Bluetooth, and ZigBee, and cellular wireless communication interfaces such as 3G and LTE, and provide compatibility in terms of various protocols used in IoT and sensor networks such as TCP, UDP, IPv6, and 6LowPAN.

The wireless communication unit 240 or 340 receives the urine or feces information from the urine- and feces-sensing unit 230 or 330, and wirelessly transmits the information to the caregiver terminal 1 using a short-range wireless communication module.

When the wireless communication unit 240 or 340 is paired with the caregiver terminal 1, the wireless communication unit 240 or 340 may transmit the urine and feces information input from the urine- and feces-sensing unit 230 or 330, and the battery remaining power information to the caregiver terminal 1, and output the confirmation signal received from the caregiver terminal 1 to the urine- and feces-sensing unit 230 or 330.

A manipulator 250 or 350 may include a switch, an LED, and the like. When the switch is turned on through the user's manipulation, the power of the battery 260 or 360 is supplied to internal components, namely, the conductive sensing line 110, the electrode-receiving portions 122 and 124, the urine- and feces-sensing unit 230 or 330, and the wireless communication unit 240 or 340 to activate the urine- and feces-sensing device. When the switch is turned off through the user's manipulation, the power supply from the battery 260 or 360 is cut off to stop the operation of the urine- and feces-sensing device.

In this connection, the manipulator 250 or 350 may turn on the LED included therein whenever the switch is turned on/off, thereby to allow the user to identify a current state of the urine- and feces-sensing device.

The battery 260 or 360 may supply power to the conductive sensing line 110, the electrode-receiving portions 122a/b and 124a/b, the urine- and feces-sensing unit 230 or 330, the wireless communication unit 240 or 340 and the manipulator 250 or 350 and activate each of the components.

According to an embodiment of the present disclosure, the urine- and feces-sensing device 430 may further include a wireless charger 270. The wireless charger 270 may be provided inside the main body 432 to charge the battery 260 or 360 in a wireless charging scheme. The wireless charger 270 may wirelessly charge the battery 260 or 360 using a wireless charger for a smartphone as widely used.

Furthermore, in this embodiment, when the battery 260 or 360 of the urine- and feces-sensing device 430 is charged in a wireless charging scheme, there is no need to install the charging port 430P for the wired charging on the main body 432. Thus, the main body 432 of the urine- and feces-sensing device 430 may be embodied in a sealed manner to have a waterproof function.

Hereinafter, interaction between the urine- and feces-sensing device according to embodiments of the present disclosure and a caregiver terminal associated therewith will be described.

First, when the user turns on the switch of the manipulator 250 or 350, the battery 260 or 360 supplies power to the urine- and feces-sensing device to turn on the urine- and feces-sensing device. The urine- and feces-sensing device connects to the caregiver terminal 1.

As described above, in the present embodiment, the wireless communication according to the Bluetooth communication scheme is performed between the urine- and feces-sensing device and the caregiver terminal 1. When the urine- and feces-sensing device is turned on, pairing between the urine- and feces-sensing device and the caregiver terminal 1 is performed.

In one example, when the urine- and feces-sensing device is turned on, a resistance value of the conductive sensing line 110 is measured at a certain time period using the urine- and feces-sensing unit 230 or 330. The measured resistance value is compared with a reference value to detect whether urine or feces is present.

When urine or feces is present, the urine- and feces-sensing device may create urine and feces information and transmit the same to the caregiver terminal 1. At this time, the urine- and feces-sensing device may measure a remaining power of the battery 260 or 360 to create battery level information, and transmit the battery level information together with the urine or feces information.

In one example, the caregiver terminal 1 continuously monitors whether the urine or feces information has been received from the urine- and feces-sensing device. The caregiver terminal 1 determines that urine or feces have been sensed when the urine and feces information has been received.

The caregiver terminal 1 may be implemented as a smart phone having an application program therein that may use a service of the present disclosure. In this case, the caregiver terminal 1 may perform urine- and feces-sensing notification in a scheme such as vibration, alarm, special sound effect, and periodic flashing of a LED lamp.

In one example, the caregiver terminal 1 checks whether the caregiver has recognized the urine- and feces-sensing, and repeats the above process when the caregiver does not input a notification release command. When the caregiver inputs the command to release the notification, the caregiver terminal 1 creates a confirmation signal and transmits the same to the urine- and feces-sensing device.

The urine- and feces-sensing device checks whether the confirmation signal (ACK signal) is received from the caregiver terminal 1, and transmits the urine and feces information again when the confirmation signal is not received even after a predetermined time has elapsed. When the confirmation signal is received by the urine- and feces-sensing device, the urine- and feces-sensing device ends the sensing operation.

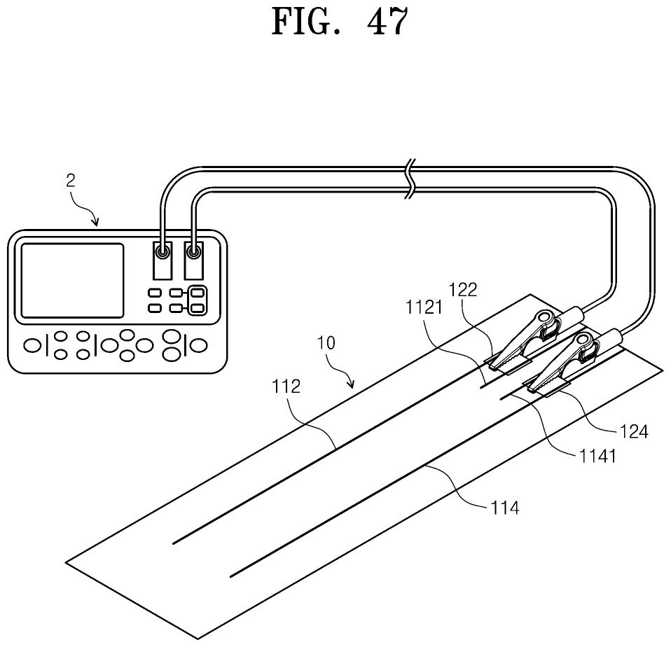

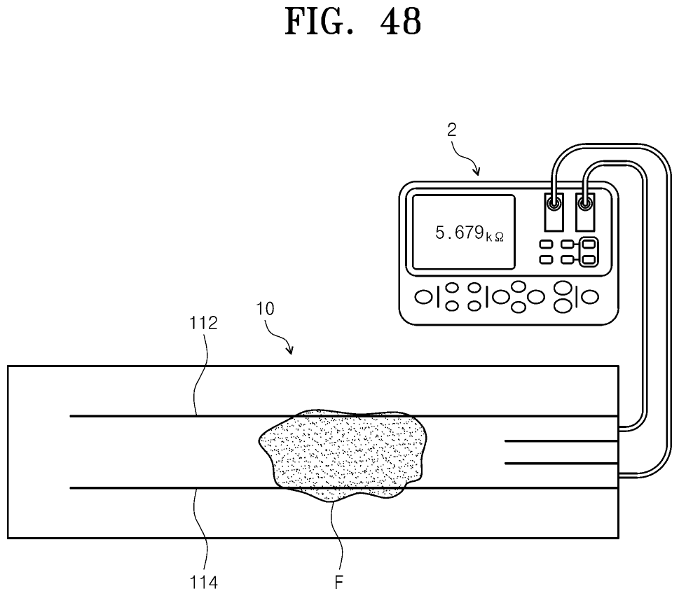

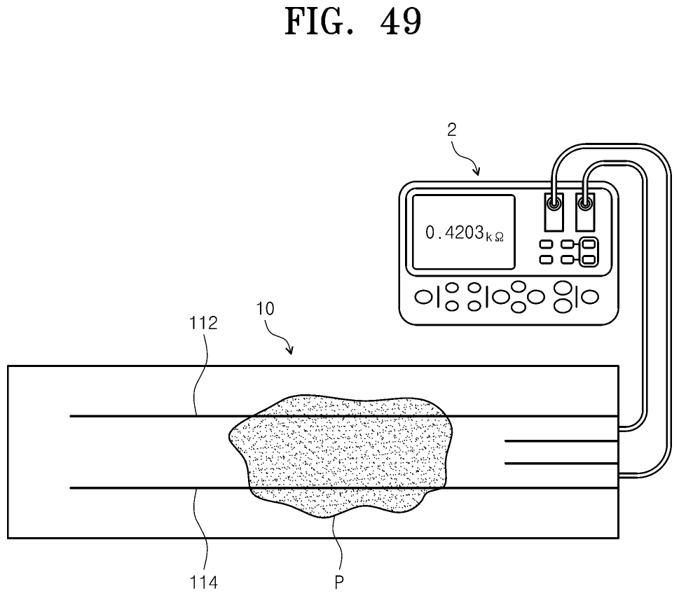

FIG. 47 is a view showing a state of sensing absence or presence of urine or feces using the urine- and feces-sensing sheet for sensing urine or feces according to an embodiment of the present disclosure. FIG. 48 is a diagram showing an example of a resistance value measured when sensing feces in one embodiment of the present disclosure. FIG. 49 is a diagram showing an example of a resistance value measured when sensing urine in one embodiment of the present disclosure.

Referring to FIG. 47, a measuring device 2 for detecting change in resistance at the electrode-receiving portions 122 and 124 may be connected with a urine and feces receiving portion to detect the absence or presence of urine or feces.

Specifically, an end of a cable connected to the measuring device 2 may be implemented in a form of tongs which may pick up the electrode-receiving portions 122 and 124.

Accordingly, the resistance in the conductive sensing line may be measured by the measuring device 2 through each of the electrode-receiving portions 122 and 124.

For example, as shown in FIG. 48, when applying a feces substitute onto the region between the conductive sensing lines 112 and 114 arranged on the lining layer 10′, a resistance value of 5.679 kilohm (kΩ) may be measured.

Moreover, as shown in FIG. 49, when dropping a urine substitute onto the conductive sensing lines 112 and 114 arranged on the lining layer 10′, a resistance value of 0.4203 kilohm (kΩ) which is a smaller resistance value than that when the feces substitute is applied may be measured.

In other words, when using the urine- and feces-sensing device of the present disclosure, not only the urine, but also the feces may be detected. The resistance value when the feces substitute is present is relatively higher than that when the urine substitute is present.

In the present embodiments, a case in which the resistance between the electrode-receiving portions 122 and 124 is measured using a resistance measuring device has been described. However, the present disclosure is not limited thereto. Capacitance measuring means may be used to measure capacitance between patch parts or inductance measuring means may be used to measure inductance between patch parts.

Thus, according to the embodiment of the present disclosure, the absence or presence of the urine or feces may be sensed in a distinguishing manner between feces and urine by detecting the electrical changes when the urine or the feces contact the lining layer or the urine- and feces-sensing sheet provided with the conductive sensing line and the electrode-receiving portion.

Further, according to one embodiment of the present disclosure, immediate urine- and feces-sensing is possible, so that the lining layer of the diaper does not need to retain a lot of moisture for a long time. Thus, development of the diaper that is more economical and human-friendly may be realized.

Hereinafter, a method of manufacturing the diaper 10 for sensing urine or feces according to embodiments of the present disclosure will be described.

In an embodiment of the present disclosure, first, the waterproof cover, the absorbing layer and the lining layer are prepared. The lining layer is made to be longer than the absorbing layer or a cover sheet. A conductive sensing line is formed on the inner surface thereof and an electrode-receiving portion is formed on one end of an outer surface. Next, the waterproof cover, the absorbing layer and the lining layer are combined with each other in this order, and the distal end of the lining layer on which the electrode-receiving portion is formed is folded outwardly from a waterproof cover boundary and attached to the waterproof cover so that the electrode-receiving portion is exposed to the outer surface of the waterproof cover.

In another embodiment, a conventional diaper composed of the waterproof cover, the absorbing layer and the lining layer is provided. Then, the conductive sensing line and the electrode-receiving portion penetrating from the lining layer of the diaper to the waterproof cover thereof are formed through a sewing process. A waterproof tape is attached to at least a portion of the conductive sensing line exposed to the outer surface of the waterproof cover to prevent leakage of urine or feces.

A specific embodiment according to the present disclosure has been described. Various modifications thereto are possible without departing from the scope of the present disclosure. Therefore, the scope of the present disclosure should not be limited to the described embodiment and should be determined not only by the scope of the claims described below, but also by the equivalents of the claims.

As described above, the present disclosure has been described based on the limited embodiments and drawings. The present disclosure is not limited to the above embodiments and may be modified and modified in various ways by those skilled in the art to which the present disclosure belongs. Therefore, idea of the present disclosure should be understood only by the claims set out below. All of its equivalent or equivalent modifications will fall within the scope and idea of the present disclosure.

1. A diaper for sensing urine and feces, including a waterproof cover, an absorbing layer located on the inner side of the waterproof cover, and a lining layer located on the inner side of the absorbing layer,
wherein the lining layer includes multiple conductive sensing lines positioned on the inner surface of the lining layer that makes contact with the human body, the conductive sensing lines being arranged separately from each other,
wherein at least one of the electrical resistance, capacitance, and inductance between the conductive sensing lines changes upon contact between the conductive sensing lines and urine or feces.

2. The diaper of claim 1, wherein each of the conductive sensing lines includes a material with electrical conductivity, has the form of a thread or a band, and is formed on the lining layer using a sewing process, an adhesion process, or a printing process.

3. The diaper of claim 1, wherein the distal end of each of the conductive sensing lines is coupled and electrically connected to a conductive electrode-receiving portion formed on the lining layer or the waterproof cover, the distal end of each of the conductive sensing lines being coupled to the conductive electrode-receiving portion by a sewing process using a conductive thread, a binding process using a conductive thread, an adhesion process using a conductive adhesive, or a combination thereof.

4. The diaper of claim 3, wherein the electrode-receiving portion includes woven fabric, knitted material, felt, or non-woven material composed of conductive fibers.

5. The diaper of claim 3, wherein one end of the lining layer on which the electrode-receiving portion is formed is folded toward and attached to the outer surface of the waterproof cover so that the electrode-receiving portion is exposed to the outer surface of the waterproof cover.

6. The diaper of claim 3, wherein the electrode-receiving portion is formed on the outer portion of the waterproof cover, wherein one end of each of the conductive sensing lines formed on the lining layer is electrically connected to the electrode-receiving portion by a sewing process using a conductive thread, a binding process using a conductive thread, an adhesion process using a conductive adhesive, or a combination thereof.

7. The diaper of claim 3, wherein the conductive sensing lines include a first sensing line and a second sensing line,
wherein the electrode-receiving portion includes a first electrode-receiving portion and a second electrode-receiving portion.

8. The diaper of claim 7, wherein the end of the first electrode-receiving portion is fixed to the lining layer or the waterproof cover by a sewing process or an adhesion process such that a space for receiving an electrode of the urine- and feces-sensing device is defined between the fixed ends of the first electrode-receiving portion,
wherein the end of the second electrode-receiving portion is fixed to the lining layer or the waterproof cover by a sewing process or an adhesion process such that a space for receiving an electrode of the urine- and feces-sensing device is defined between the fixed ends of the second electrode-receiving portion.

9. The diaper of claim 3, wherein the conductive sensing lines include a first sensing line and a second sensing line,
wherein the electrode-receiving portion includes a single fourth electrode-receiving portion,
wherein the two ends of the fourth electrode-receiving portion are respectively connected to the first and second sensing lines by a sewing process using a conductive thread, a binding process using a conductive thread, an adhesion process using a conductive adhesive, or a combination thereof, such that a space for receiving an electrode of the urine- and feces-sensing device is defined between the fixed ends of the fourth electrode-receiving portion.

10. The diaper of claim 9, wherein the fourth electrode-receiving portion includes a first conductive cloth connected to the first sensing line, a second conductive cloth connected to the second sensing line, and a non-conductive connector connecting the first conductive cloth with the second conductive cloth separated from the first.

11. The diaper of claim 1, wherein the conductive sensing lines include multiple unit-sensing lines implemented with an electrical connection and spaced apart from each other.

12. (canceled)

13. (canceled)

14. (canceled)

15. A urine- and feces-sensing sheet attached to the inner surface of the diaper to give the diaper a urine- and feces-sensing function to the diaper, the diaper including a waterproof cover, an absorbing layer, and a lining layer,
wherein the urine- and feces-sensing sheet includes multiple conductive sensing lines attached to the lining layer and positioned on the inner surface of the urine- and feces-sensing sheet that makes contact with the human body, the conductive sensing lines being arranged separately from each other,
wherein at least one of the electrical resistance, capacitance, and inductance between the conductive sensing lines changes upon contact between the conductive sensing lines and urine or feces.

16. The urine- and feces-sensing sheet of claim 15, wherein the urine- and feces-sensing sheet is composed of the same material as the lining layer or a material with absorbency power corresponding to the lining layer,
wherein the urine- and feces-sensing sheet includes an attachment feature on an attachment surface that makes contact with the lining layer and is attached to the lining layer.

17. The urine- and feces-sensing sheet of claim 15, wherein each of the conductive sensing lines includes a material with electrical conductivity, has the form of a thread or a band, and is formed on the urine- and feces-sensing sheet using a sewing process, an adhesion process, or a printing process.

18. The urine- and feces-sensing sheet of claim 15, wherein the distal end of each of the conductive sensing lines is coupled to and is electrically connected to a conductive electrode-receiving portion formed on the urine- and feces-sensing sheet, the distal end of each of the conductive sensing lines being coupled to the conductive electrode-receiving portion by a sewing process using a conductive thread, a binding process using a conductive thread, an adhesion process using a conductive adhesive, or a combination thereof.

19. The urine- and feces-sensing sheet of claim 18, wherein the electrode-receiving portion includes woven fabric, knitted material, felt, or non-woven material composed of conductive fibers.

20. The urine- and feces-sensing sheet of claim 18, wherein one end of the urine- and feces-sensing sheet on which the electrode-receiving portion is formed is folded toward and attached to the outer surface of the waterproof cover of the diaper so that the electrode-receiving portion is exposed to the outer surface of the waterproof cover.

21. The urine- and feces-sensing sheet of claim 18, wherein the conductive sensing lines include a first sensing line and a second sensing line,
wherein the electrode-receiving portion includes a first electrode-receiving portion and a second electrode-receiving portion.

22. The urine- and feces-sensing sheet of claim 21, wherein the end of the first electrode-receiving portion is fixed to the urine- and feces-sensing sheet by a sewing process or an adhesion process such that a space for receiving an electrode of the urine- and feces-sensing device is defined between the fixed ends of the first electrode-receiving portion,
wherein the end of the second electrode-receiving portion is fixed to the urine- and feces-sensing sheet by a sewing process or an adhesion process such that a space for receiving an electrode of the urine- and feces-sensing device is defined between the fixed ends of the second electrode-receiving portion.

23. The urine- and feces-sensing sheet of claim 18, wherein the conductive sensing lines include a first sensing line and a second sensing line, and
the electrode-receiving portion includes a single fourth electrode-receiving portion,
wherein the two ends of the fourth electrode-receiving portion are respectively connected to the first and second sensing lines by a sewing process using a conductive thread, a binding process using a conductive thread, an adhesion process using a conductive adhesive, or a combination thereof, such that a space for receiving an electrode of the urine- and feces-sensing device is defined between the fixed ends of the fourth electrode-receiving portion.

24. The urine- and feces-sensing sheet of claim 23, wherein the fourth electrode-receiving portion includes a first conductive cloth connected to the first sensing line, a second conductive cloth connected to the second sensing line, and a non-conductive connector connecting the first conductive cloth with the second conductive cloth separated from the first.

25. The urine- and feces-sensing sheet of claim 17, wherein the conductive sensing lines include multiple unit-sensing lines implemented with an electrical connection and spaced apart from each other.

26. (canceled)

27. (canceled)

28. (canceled)

29. A urine- and feces-sensing device connected to a urine- and feces-sensing diaper or to a urine- and feces-sensing sheet for a diaper to sense urine and feces based on at least one of the electrical resistance, capacitance, and inductance associated with at least one conductive sensing line positioned on the urine- and feces-sensing diaper or sheet, comprising:
a urine- and feces-sensing unit for sensing absence or presence of the urine or feces of a diaper wearer based on at least one of the electrical resistance, capacitance, and inductance;
a main body accommodating the urine- and feces-sensing unit;
an electrode connected to the main body and inserted into a space defined in the electrode-receiving portion formed on the urine- and feces-sensing diaper or sheet; and
a cover coupled to the main body through a rotatable hinge to pivot in a predetermined angle range for opening and closing, the cover being configured to fasten the electrode to the electrode-receiving portion.

30. The device of claim 29, wherein an electrode contact is formed on the top surface of the electrode by plating a conductive material on the electrode, ensuring that the electrode contact protrudes to a predetermined height.

31. The device of claim 30, wherein the electrode contact is electrically connected to the urine- and feces-sensing unit.

32. The device of claim 29, wherein a magnet is installed at one end of the top surface of the electrode and a magnet is installed at one end of the bottom surface of the cover facing the top surface of the electrode, such that the closed state of the cover relative to the main body is maintained through attracting force between the magnets.

33. The device of claim 29, wherein a spring and a soft spring cover are installed in a space defined on the bottom surface of the cover, such that the pressing force from the spring is maintained in the region where the electrode contact and the electrode-receiving portion overlap with each other while the electrode is inserted into the electrode-receiving portion and the cover is closed.

34. The device of claim 29, further including a power switch button for powering the main body on or off.

35. The device of claim 29, wherein the urine- and feces-sensing unit wirelessly transmits urine- and feces-sensing information to a caregiver terminal or a relay communicating with the caregiver terminal.
